(19) United States
(12) Patent Application Publication
(10) Pub. No.: US 2025/0325712 A1
(43) Pub. Date: Oct. 23, 2025

(54) TARGETING LIGANDS FOR DISEASE-TARGETED IMAGING AGENTS AND METHODS OF USE THEREFOR

(71) Applicant: Curadel Surgical Innovations, Inc., Natick, MA (US)

(72) Inventor: John V. Frangioni, Wayland, MA (US)

(21) Appl. No.: 18/641,097

(22) Filed: Apr. 19, 2024

(51) Int. Cl. A61K 49/00; A61K 51/08; A61P 35/00

(52) U.S. Cl. CPC A61K 49/0056; A61K 49/0032; A61K 51/088; A61P 35/00

(57) ABSTRACT

In accordance with at least one aspect of this disclosure, there is provided an imaging agent, a including dye compound conjugated to an antigen specific targeting vector which can be particularly advantageous because their behavior in vivo can contribute to superior optical imaging properties, for example, by significantly increasing the target-to-background ratio of imaged tissues, leading to higher resolution imaging, and ultimately providing for better recognition of malignant tissue for resection and margin assessment and improved visualization during minimal invasive laparoscopic surgery, for example. A method of imaging tumor cells in a subject, can include, administering an imaging effective amount of an imaging agent according at least one embodiment of the invention; 2-4 hours after administration of the imaging agent, irradiating a region in the subject in which tumor cells are expected to be found with at a wavelength absorbed by the imaging agent; and detecting a signal from the imaging agent, thereby imaging the tumor cells, wherein the target-to-background ratio 2-4 hours after administration is from about 2.75 to about 15.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Apr. 17, 2024, is entitled “1515138106US0_SL.xml”, and is 8,192 bytes in size.

### FIELD OF THE INVENTION

The present invention relates to methods of optically imaging tissues or cells using zwitterionic imaging agents having desirable in vivo properties that result in improved target-to-background ratio (hereinafter “TBR”) as compared to other near-infrared (NIR) contrast agents.

### BACKGROUND OF THE INVENTION

Near infrared (NIR) fluorescence has potential importance in the medical field, particularly in diagnostics and image-guided surgery. However, the availability of suitable fluorophores as imaging agents has been a primary hindrance. To be clinically viable, the ideal NIR fluorophore should have both good optical properties and superior in vivo properties with respect to solubility, metabolism, biodistribution, and clearance. Known fluorophores tend to clear through the liver, which results in undesirable fluorescence throughout the gastrointestinal tract. And in some cases, known fluorophores suffer from significant non-specific background uptake in normal tissues, resulting in a low target-to-background ratio (TBR).

In recent years, more advanced fluorophores have been developed which result in improved target-to-background. See, for example, U.S. Pat. Nos. 11,077,210, 9,687,567, 10,493,169, 10,201,621, and 10,478,512. Although these advanced fluorophores lower non-specific uptake in non-target tissue, they are relatively labile in blood, becoming metabolized in minutes to hours. As such, there remains a need for new and improved NIR fluorescent imaging agents with tumor- or diseased-tissue targeting and/or increased stability that can equilibrate rapidly between the intravascular and extravascular spaces and are cleared efficiently, including by renal filtration.

Similarly, although prior fluorophores have had some success in targeting specific antigens and other markers which are expressed by a variety of cancer cell types, there remains a need for increased targeting of specific antigens to increase the specific uptake of the fluorophores to increase signal strength. For example, targeting vectors directed to prostate-specific membrane antigen (PSMA), bombesin receptors, somatostatin receptors, fibroblast activation protein (FAP) would provide fluorophores with increased signal for more accurate imaging of various tumors and benign but diseased tissues, as well as tumor stroma, and other diseases characterized by tissue remodeling. The imaging agents of the invention are directed toward these and other needs.

An improved target-to-background provides for better recognition of malignant tissue for resection and margin assessment as well as improved visualization during minimally-invasive laparoscopic surgery. Accordingly, while certain conventional fluorophores may result in improved target-to-background ratio, there remains a need in the art for further increasing target-to-background ratios to assist practitioners in performing complete and accurate resections of cancerous tissues during surgery.

### SUMMARY OF THE INVENTION

The invention is based, at least in part, on the discovery that the inclusion of antigen specific targeting vectors can increase the target-to-background ratio of zwitterionic fluorophores without reducing the stability or clearance afforded by other elements of the fluorophore design.

The imaging agents of the invention are particularly advantageous because their behavior in vivo is believed to contribute to superior optical imaging properties and, in some cases, superior stability. More specifically, the charge-balancing is believed to impart good biodistribution and clearance properties to the agents and reduce undesirable non-specific binding while the inclusion of the targeting ligand increases the time of circulation and prevents additional degradation after binding to target cells. These in vivo properties help improve the target-to-background ratio of imaged tissues, leading to higher resolution imaging.

Other features and advantages of the invention will be apparent from the following detailed description, and from the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

In at least one aspect, the invention provides an imaging agent dye comprising a charge-balanced imaging agent conjugated to targeting vector, the imaging agent dye having a labile linkage between the imaging agent and the targeting vector. Upon cell binding and internalization into acidic environments within the target cell, the imaging agent dye is stabilized. However, external to the target cell, the labile linkage is decomposed rendering the imaging agent dye non-fluorescent and thus increasing the target cell to background ratio over time after administration of the imaging dye. In certain embodiments, the labile linkage between the imaging agent and the targeting vector is at a position closer to the imaging agent.

In certain embodiments, the charge-balanced imaging agent is ZW-800-1, or ZW-830-1, or ZW-700-1-Forte.

In certain embodiments, the targeting vector is cRGD, a PSMA binding vector, such as dPSMA-617 or KUE, a FAP binding vector, a bombesin receptor binding vector, or a somatostatin receptor binding vector.

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin

In certain embodiments, the charge-balanced imaging agent is conjugated to the targeting vector via a direct bond, or via a linking group.

In at least one aspect, the invention provides, an imaging agent comprising a conjugate having the formula:

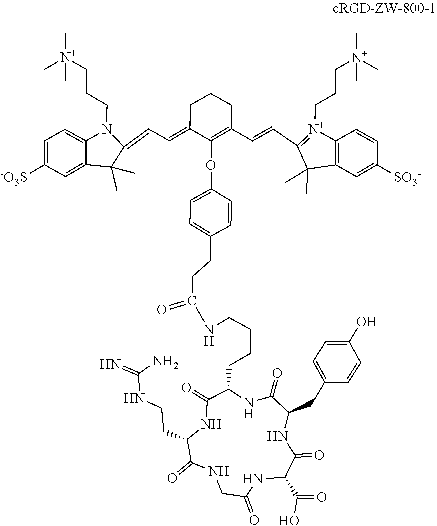

wherein L is an optional linking group;
or a salt, solvate, or hydrate thereof;
or dimer thereof.

In at least one aspect, the invention provides, an imaging agent comprising a conjugate having the formula:

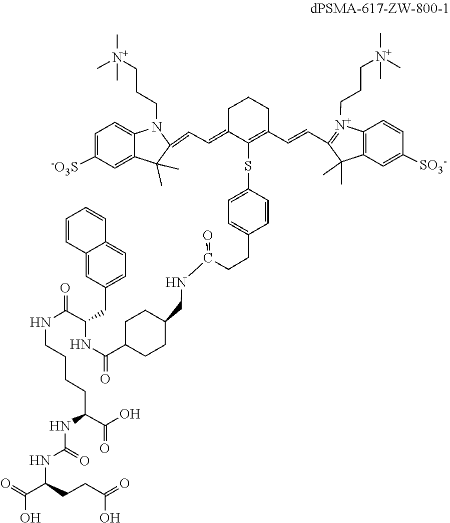

wherein L is an optional linking group;
or a salt, solvate, or hydrate thereof;
or dimer thereof.

In at least one aspect, the invention provides, an imaging agent comprising a conjugate having the formula:

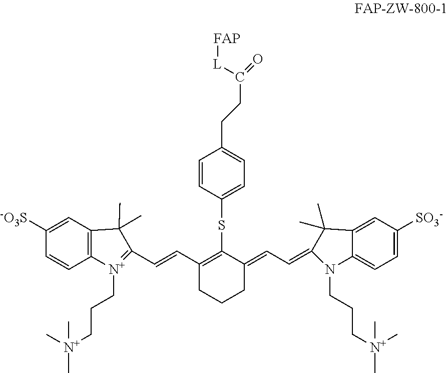

wherein L is an optional linking group;
or a salt, solvate, or hydrate thereof;
or dimer thereof.

In at least one aspect, the invention provides, an imaging agent comprising a conjugate having the formula:

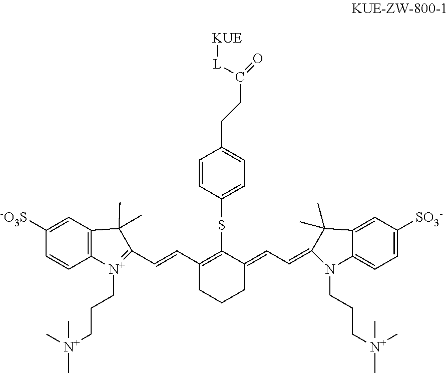

wherein L is an optional linking group;
or a salt, solvate, or hydrate thereof;
or dimer thereof.

In at least one aspect, the invention provides, an imaging agent comprising a conjugate having the formula:

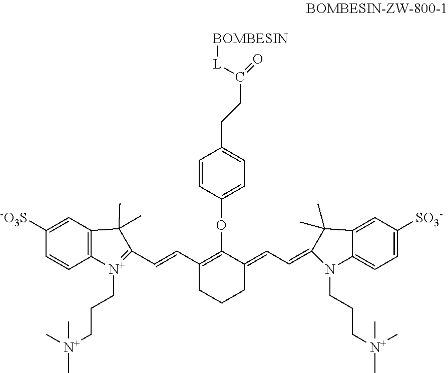

wherein L is an optional linking group;
or a salt, solvate, or hydrate thereof;
or dimer thereof.

In at least one aspect, the invention provides, an imaging agent comprising a conjugate having the formula:

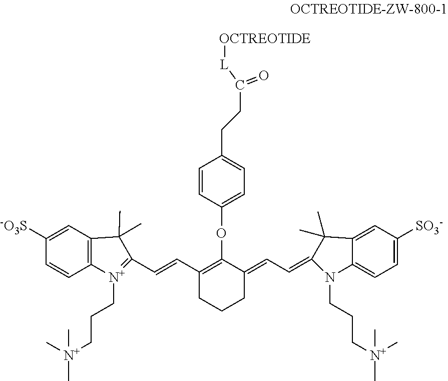

wherein L is an optional linking group;
or a salt, solvate, or hydrate thereof;
or dimer thereof.

In at least one aspect, the invention provides, an imaging agent comprising a conjugate having the formula:

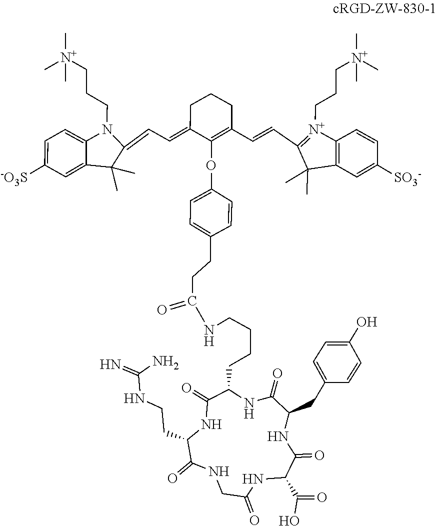

wherein L is an optional linking group;
or a salt, solvate, or hydrate thereof;
or dimer thereof.

In at least one aspect, the invention provides, an imaging agent comprising a conjugate having the formula:

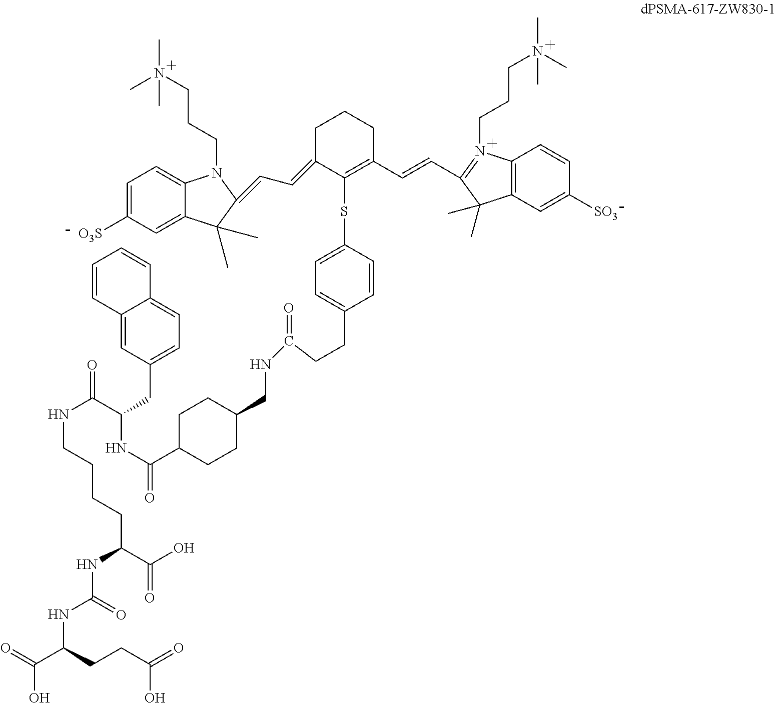

wherein L is an optional linking group;
or a salt, solvate, or hydrate thereof;
or dimer thereof.

In at least one aspect, the invention provides, an imaging agent comprising a conjugate having the formula:

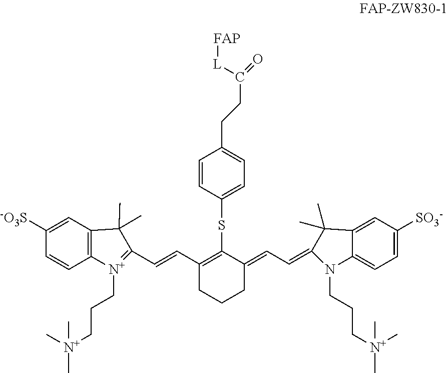

wherein L is an optional linking group;
or a salt, solvate, or hydrate thereof;
or dimer thereof.

In at least one aspect, the invention provides, an imaging agent comprising a conjugate having the formula:

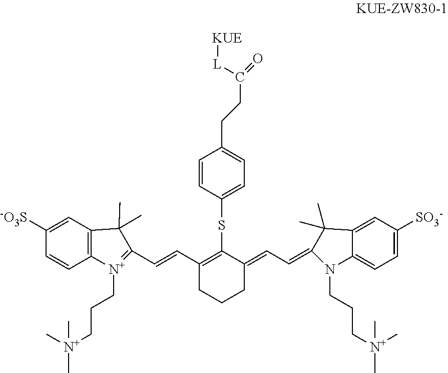

wherein L is an optional linking group;
or a salt, solvate, or hydrate thereof;
or dimer thereof.

In at least one aspect, the invention provides, an imaging agent comprising a conjugate having the formula:

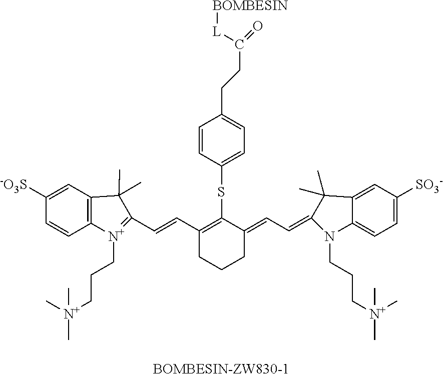

wherein L is an optional linking group;
or a salt, solvate, or hydrate thereof;
or dimer thereof.

In at least one aspect, the invention provides, an imaging agent comprising a conjugate having the formula:

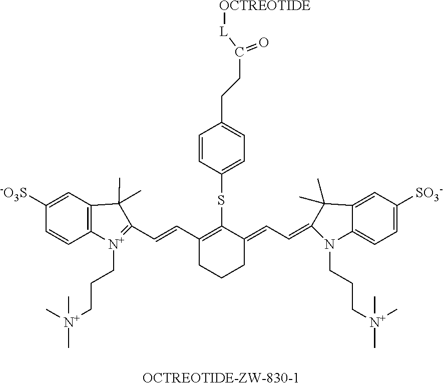

wherein L is an optional linking group;
or a salt, solvate, or hydrate thereof;
or dimer thereof.

In at least one aspect, the invention provides, an imaging agent comprising a conjugate having the formula:

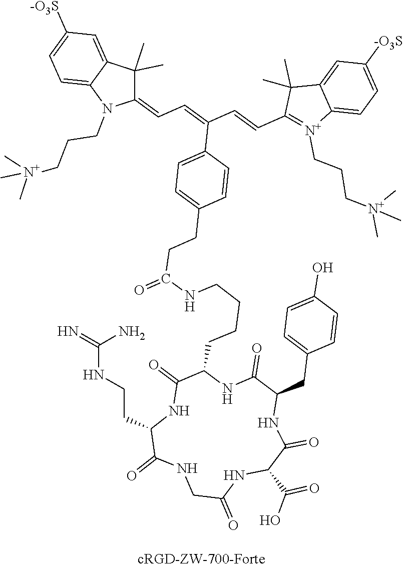

wherein L is an optional linking group;
or a salt, solvate, or hydrate thereof;
or dimer thereof.

In at least one aspect, the invention provides, an imaging agent comprising a conjugate having the formula:

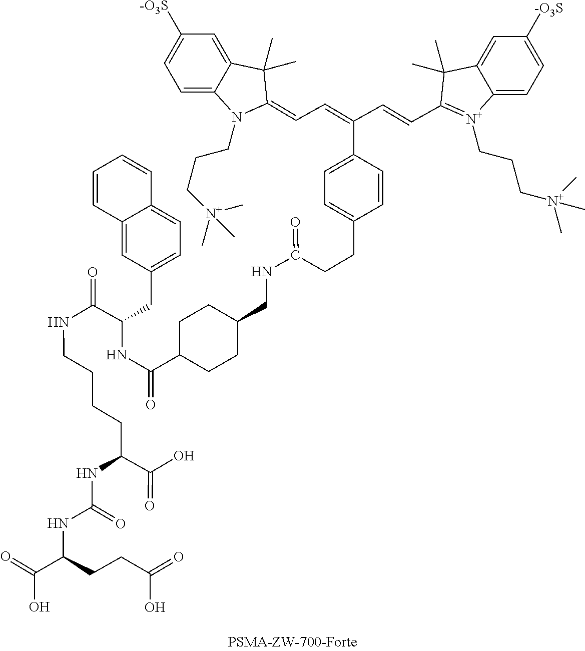

wherein L is an optional linking group;
or a salt, solvate, or hydrate thereof;
or dimer thereof.

In at least one aspect, the invention provides, an imaging agent comprising a conjugate having the formula:

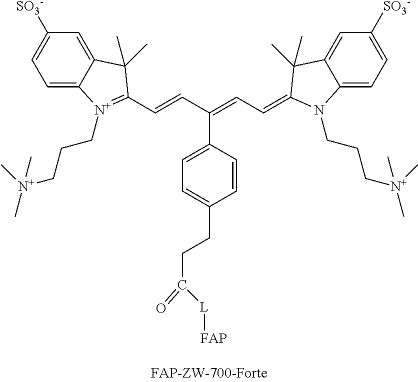

wherein L is an optional linking group;
or a salt, solvate, or hydrate thereof;
or dimer thereof.

In at least one aspect, the invention provides, an imaging agent comprising a conjugate having the formula:

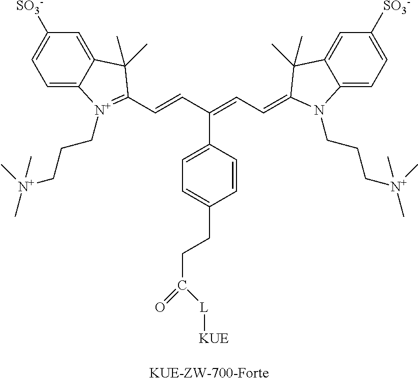

wherein L is an optional linking group;
or a salt, solvate, or hydrate thereof;
or dimer thereof.

In at least one aspect, the invention provides, an imaging agent comprising a conjugate having the formula:

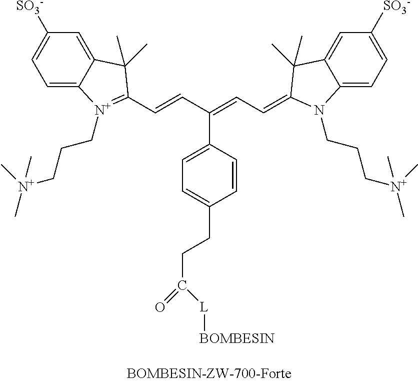

wherein L is an optional linking group;
or a salt, solvate, or hydrate thereof;
or dimer thereof.

In at least one aspect, the invention provides, an imaging agent comprising a conjugate having the formula:

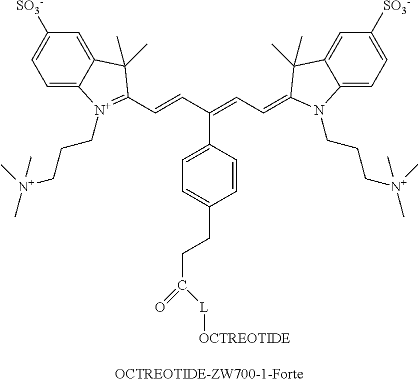

wherein L is an optional linking group;
or a salt, solvate, or hydrate thereof;
or dimer thereof.

In at least one aspect, the invention provides, a method of imaging cells, e.g., tumor cells in a subject. In certain embodiments, the method includes, administering an imaging effective amount of an imaging agent according to any one or more embodiments of the invention described herein to the subject; 2-4 hours after administration of the imaging agent, irradiating a region in the subject in which tumor cells are expected to be found with at a wavelength absorbed by the imaging agent; and detecting a signal from the imaging agent, thereby imaging the tumor cells, where the target-to-background ratio 2-24 hours after administration is from about 2.75 to about 15.

In certain embodiments, the target-to-background ratio 2-24 hours after administration is from about 2.75 to about 15.

In certain embodiments, the target-to-background ratio 2-24 hours after administration is from about 3 to about 12.5.

In certain embodiments, the target-to-background ratio 2-24 hours after administration is from about 4 to about 10.

In certain embodiments, the target-to-background ratio 2-24 hours after administration is greater than about 4.2.

In certain embodiments, the target-to-background ratio 2-24 hours after administration is greater than about 6.2.

In certain embodiments, the target-to-background ratio 2-24 hours after administration is greater than about 7.6.

In certain embodiments, the target-to-background ratio 2-24 hours after administration is two-fold or more increase than a target-to-background ratio 2-24 hours after administration of a fluorophore without an embodiment of a targeting ligand as described herein.

In certain embodiments, the target-to-background ratios discussed above are present 2-4 hours after administration. In certain embodiments, the target-to-background ratios discussed above are present 4-10 hours after administration. In certain embodiments, the target-to-background ratios discussed above are present 12-24 hours after administration. In certain embodiments, the target-to-background ratios discussed above are present 15-18 hours after administration. In certain embodiments, the target-to-background ratios discussed above are present 18-24 hours after administration.

In certain embodiments, the imaging agent has peak absorbance at about 600 nm to 850 nm.

In certain embodiments of the method of imaging cells according to the invention, the cells are tumor cells, inflammatory cells, or cells undergoing angiogenesis. In certain embodiments of the method of imaging cells according to the invention, the imaging agent is administered to an organism comprising or suspected of comprising the cells. In certain embodiments of the method of imaging cells according to the invention, the organism is human. In certain embodiments, the tissue or cells is imaged in vivo.

In certain embodiments of the method of imaging cells according to the invention, the imaging agent further comprises a PEG-moiety.

In certain embodiments of the method of imaging cells according to the invention, the linking group is the PEG-moiety.

In certain embodiments of the method of imaging cells according to the invention, the imaging agent further comprises a radioisotope for either single-photon emission computed tomography (SPECT) or positron emission tomography (PET).

In certain embodiments of the method of imaging cells according to the invention, the imaging agent comprises a reactive linking group, such as NHS ester, sulfo-NHS ester, or a TFP ester.

In at least one aspect, the invention provides a method of treating cancer in a subject. In certain embodiments of the method according to the invention, the method includes, administering an imaging effective amount of an imaging agent according to any one or more embodiments of the invention described herein to a subject; irradiating the cells, tissues or organs of a subject suspected of being cancerous at a wavelength absorbed by the imaging agent; diagnosing the cancer in the cells tissues, or organs of the subject by detecting a signal from the imaging agent; and administering a chemotherapeutic treatment, a radiotherapeutic treatment, or a surgical treatment to the subject to treat the cancer.

Other features and advantages of the invention will be apparent from the following detailed description, and from the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

So that those skilled in the art to which the subject disclosure appertains will readily understand how to make and use the devices and methods of the subject disclosure without undue experimentation, other embodiments thereof will be described in detail herein below with reference to certain figures, wherein:

FIG. 1 a representation of a dye, targeting ligand, and conjugate imaging agent of the invention.

FIG. 2 a representation of a dye, targeting ligand, and conjugate imaging agent of the invention.

FIG. 3 a representation of a dye, targeting ligand, and conjugate imaging agent of the invention.

FIG. 4 a representation of a dye, targeting ligand, and conjugate imaging agent of the invention.

FIG. 5 a representation of a dye, targeting ligand, and conjugate imaging agent of the invention.

FIG. 6 a representation of a dye, targeting ligand, and conjugate imaging agent of the invention.

FIG. 7 a representation of a dye, targeting ligand, and conjugate imaging agent of the invention.

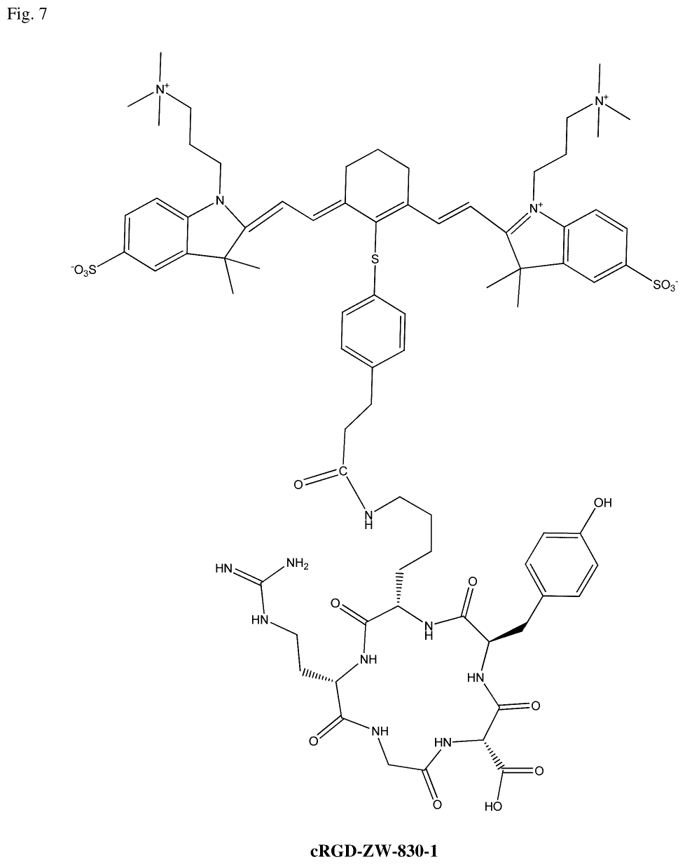

FIG. 8 a representation of a dye, targeting ligand, and conjugate imaging agent of the invention.

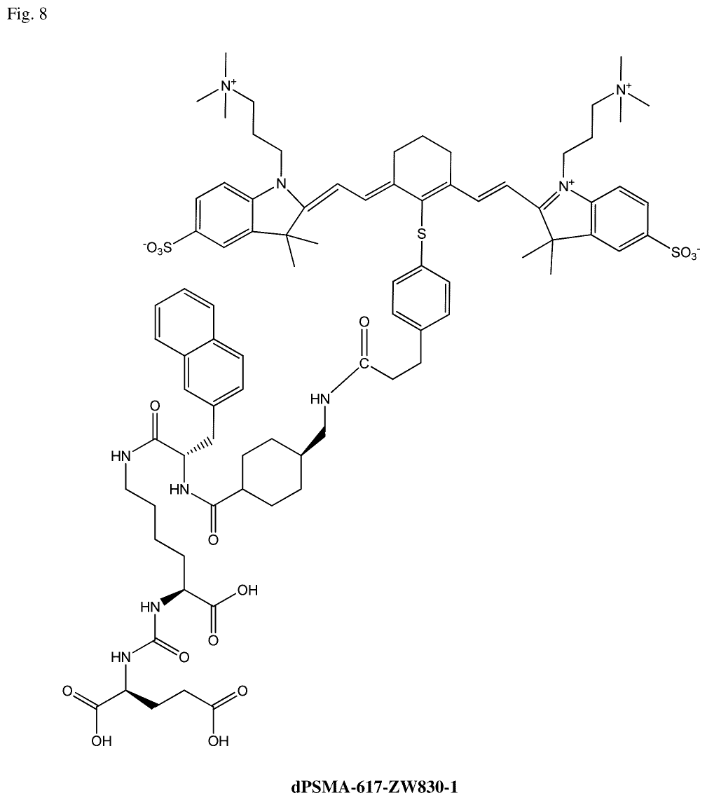

FIG. 9 a representation of a dye, targeting ligand, and conjugate imaging agent of the invention.

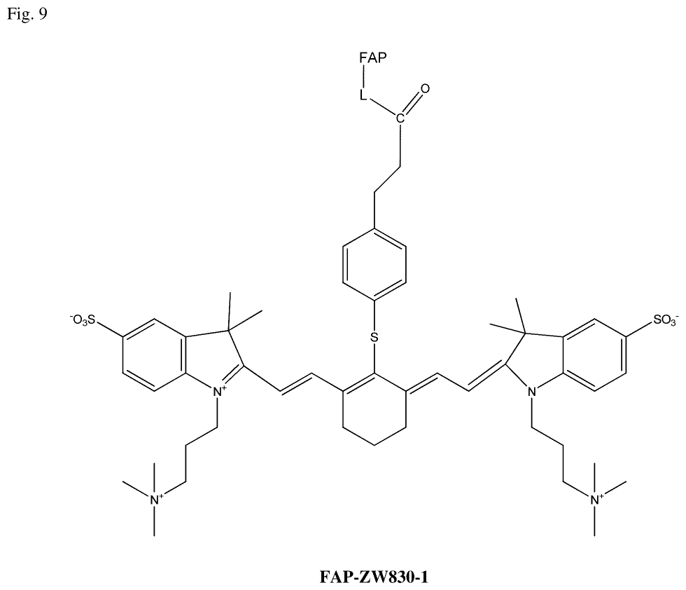

FIG. 10 a representation of a dye, targeting ligand, and conjugate imaging agent of the invention.

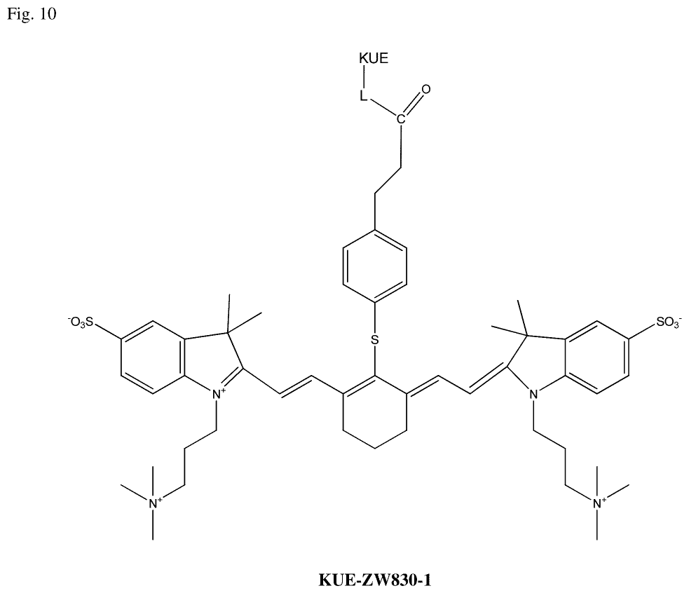

FIG. 11 a representation of a dye, targeting ligand, and conjugate imaging agent of the invention.

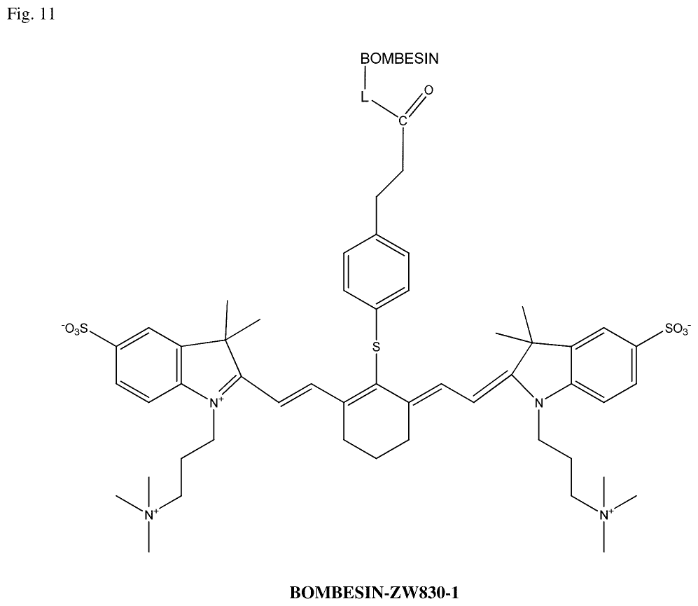

FIG. 12 a representation of a dye, targeting ligand, and conjugate imaging agent of the invention.

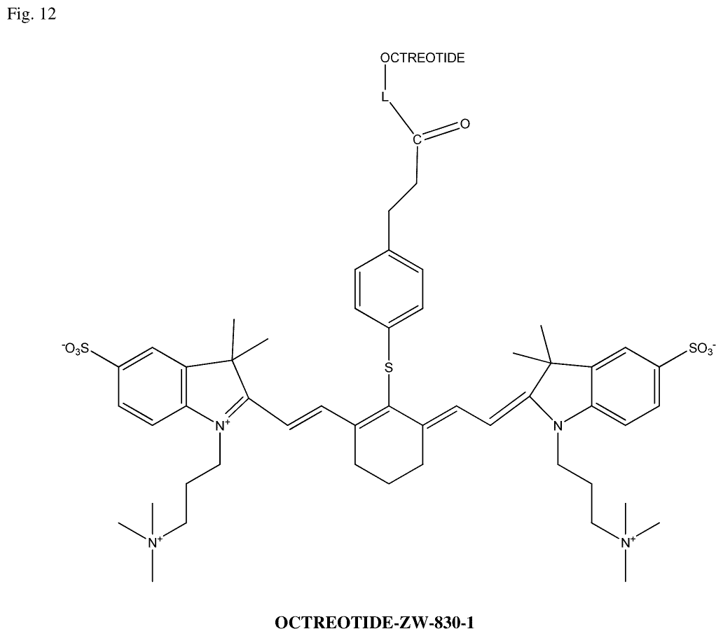

FIG. 13 a representation of a dye, targeting ligand, and conjugate imaging agent of the invention.

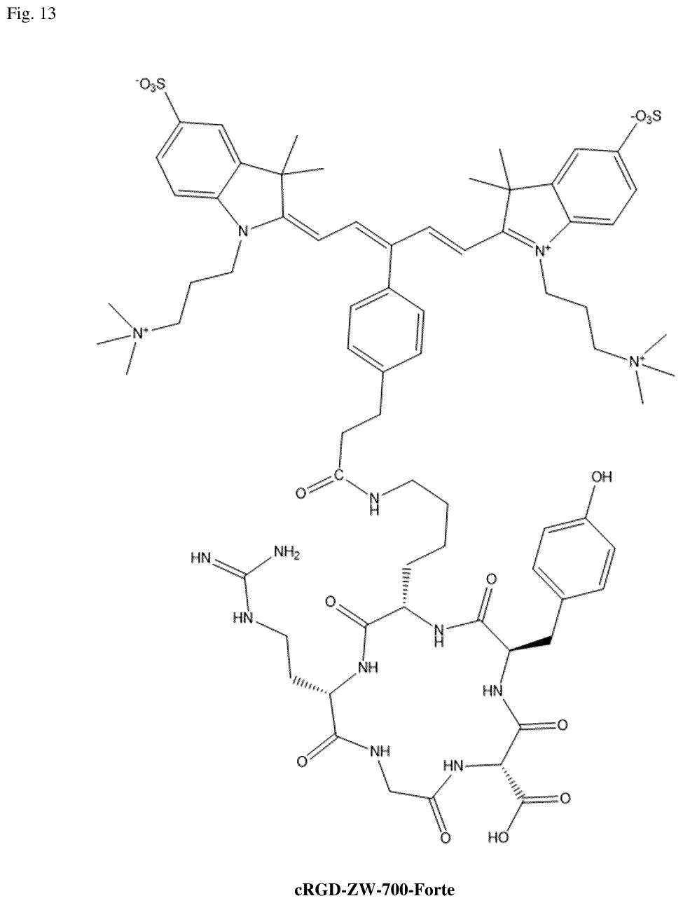

FIG. 14 a representation of a dye, targeting ligand, and conjugate imaging agent of the invention.

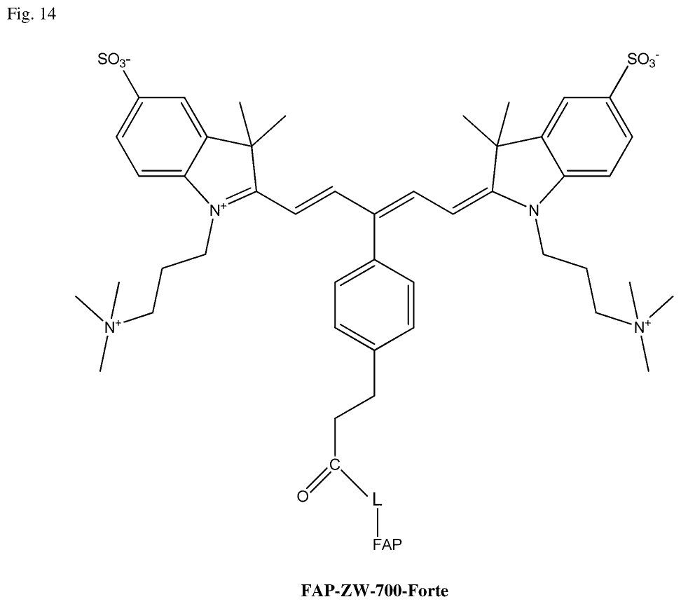

FIG. 15 a representation of a dye, targeting ligand, and conjugate imaging agent of the invention.

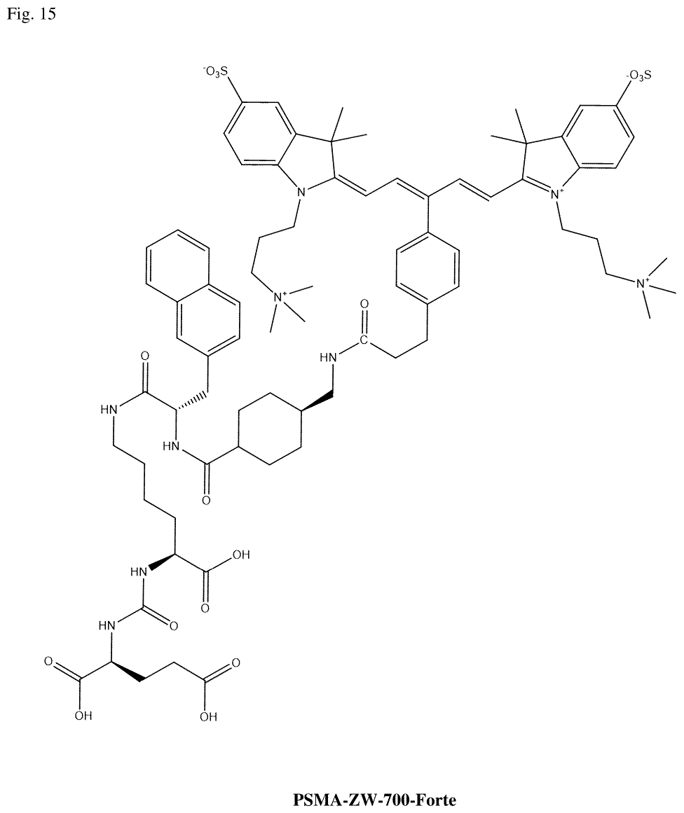

FIG. 16 a representation of a dye, targeting ligand, and conjugate imaging agent of the invention.

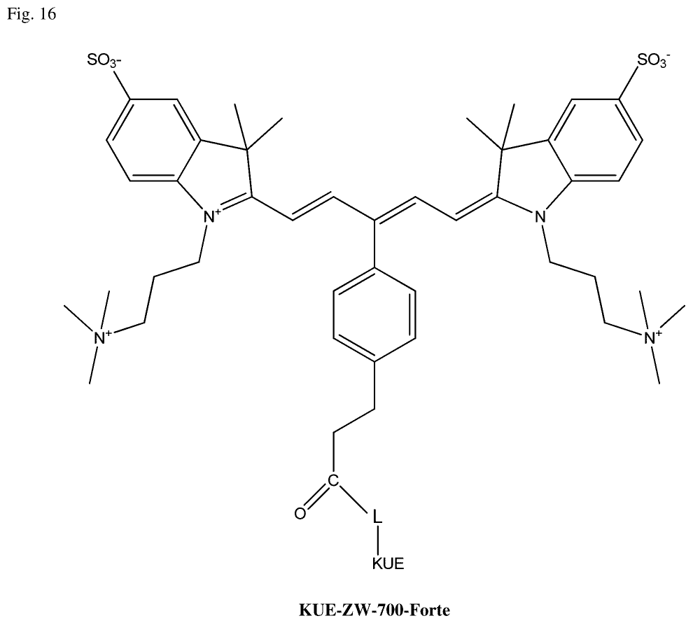

FIG. 17 a representation of a dye, targeting ligand, and conjugate imaging agent of the invention.

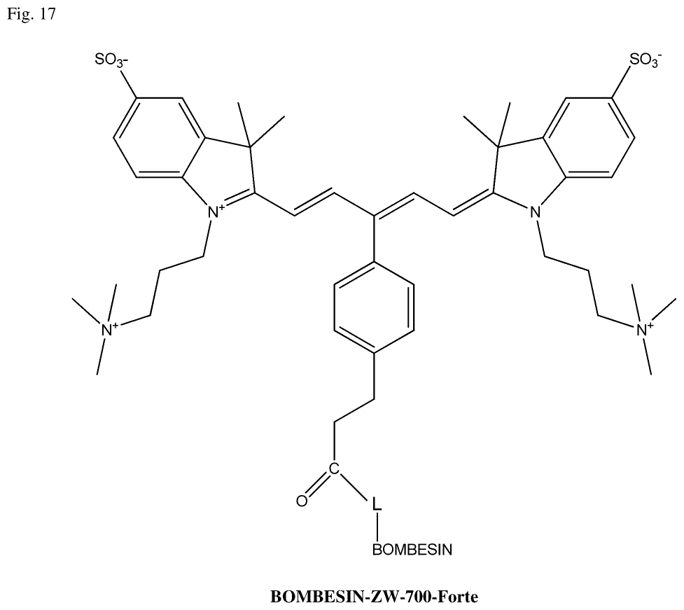

FIG. 18 a representation of a dye, targeting ligand, and conjugate imaging agent of the invention.

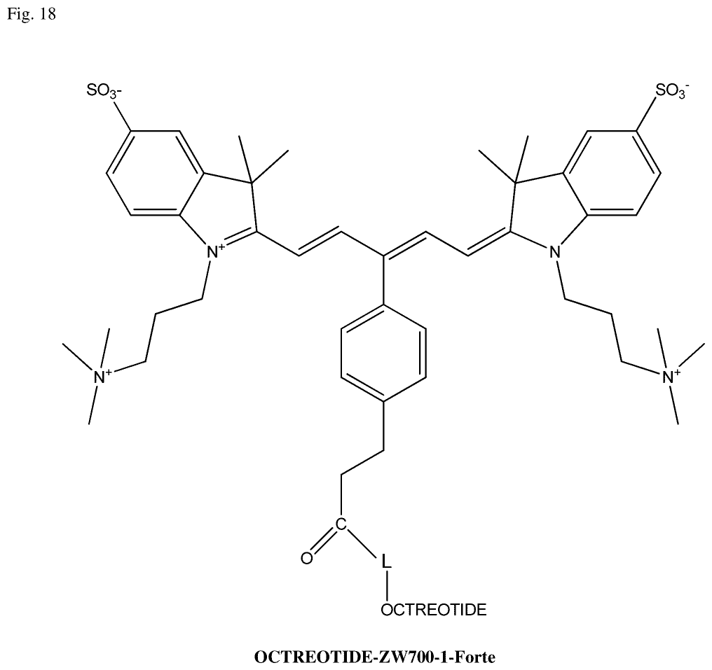

FIG. 19 shows the calculated ex vivo TBR per dose in colon tumors, in the different dose levels and injection time windows, where the stars represent a significant increase in ex vivo TBR.

FIG. 20 shows the normalized absorbance at 645 nm of ZW700-1-Forte in 37° C. buffered fetal bovine serum (FBS), pH 7.4 over time from 0 to 60 hours.

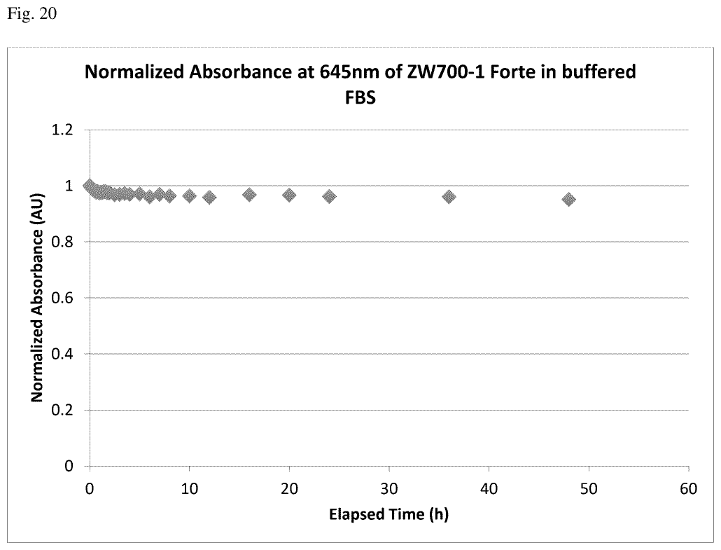

FIG. 21 shows the normalized absorbance at 768 nm of ZW800-1 in 37° C. buffered FBS, pH 7.4 over time from 0 to 60 hours.

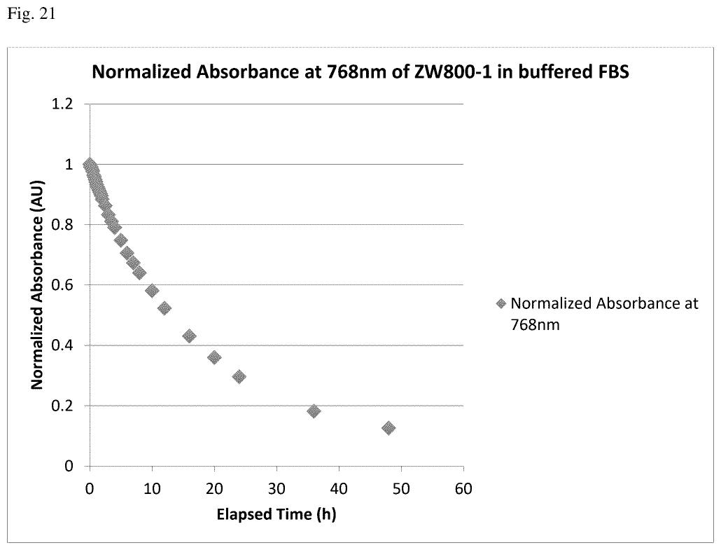

FIG. 22 shows an overlay of the data from FIGS. 20 and 21.

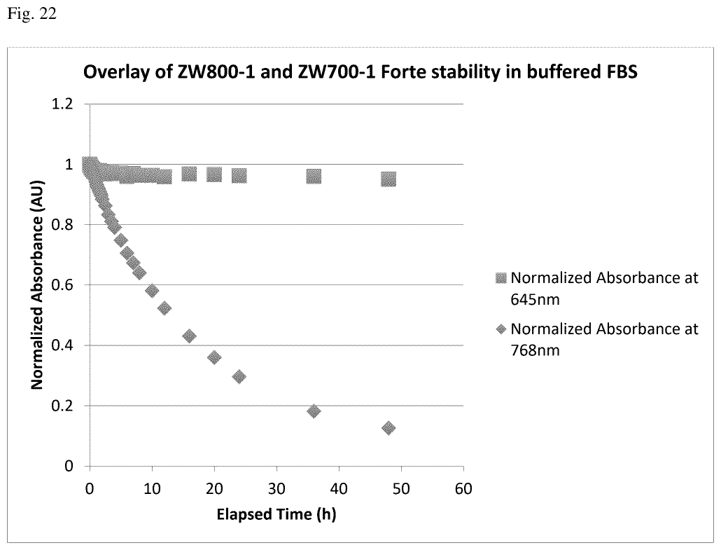

FIG. 23 shows the full absorbance spectra for ZW700-1-Forte in 37° C. buffered FBS, pH 7.4 over time from 0 to 48 hours.

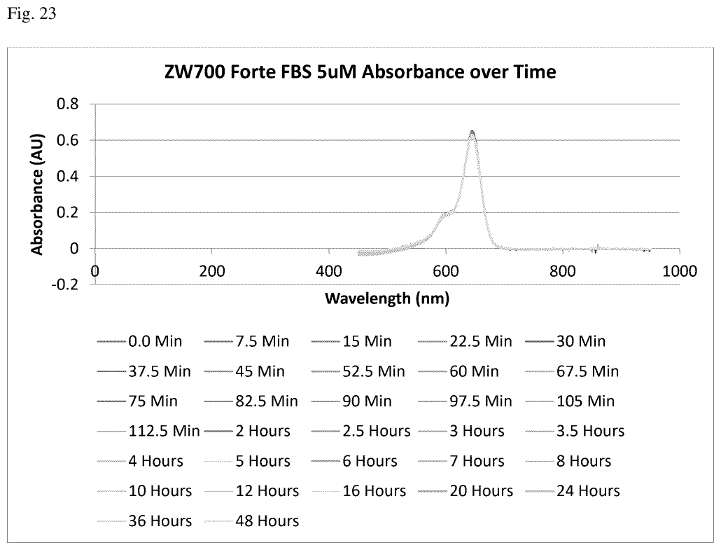

FIG. 24 shows the full absorbance spectra for ZW800-1 in 37° C. buffered FBS, pH 7.4 over time from 0 to 48 hours.

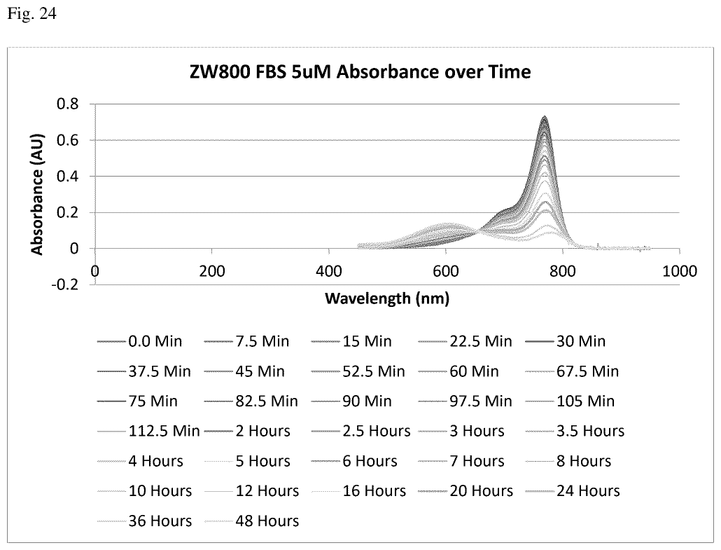

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

The present disclosure relates, inter alia, to an imaging agent that is composed of a dye molecule optionally conjugated to a targeting ligand through a linking group. The imaging agent described herein is useful in, for example, the detection of abnormal or diseased biological tissues and cells. The conjugate is particularly useful for imaging whole organisms, because it has improved in vivo behavior, such as low non-specific binding to non-targeted tissues and ultrahigh stability, resulting in an improved target-to-background ratio in connection with the detected optical signal. It is believed that these improved in vivo properties result from the balancing of formal charges on the conjugate, rendering a “charge-balanced” molecule having a net charge that is neutral or close to neutral.

### DEFINITIONS AND ADDITIONAL EMBODIMENTS

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

The following definitions will be useful in understanding the instant invention.

In some embodiments, imaging agents of the invention can also comprise a targeting vector for an agricultural process, chemical process, disease, or tissue-specific epitope, such as the cyclic peptide cRGDyK (aka cRGD) bound to the imaging agent. cRGD is a cyclic derivative of the tripeptide Arg-Gly-Asp which can be conjugated to one or more of the imaging agents of the invention. In still other embodiments, the targeting vector is octreotide or bombesin. In other embodiments, the targeting vector is KUE or dPSMA-617, a small molecule capable of targeting Fibroblast Activation Protein (FAP) also called FAP-inhibitor or FAPI, an amino acid or combination of amino acids, or derivatives thereof. In such embodiments, the targeting vector-conjugates can be formed in place of one or more zwitterionic groups. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

An ideal zwitterionic imaging dyen conjugated to a targeting vector would adopt the total net charge of the targeting vector, which is purposeful because in most cases the charges on the targeting vector are crucial for the ability to bind its target. Targeted zwitterionic imaging dyes thus retain the major advantage of minimizing non-specific binding while maximizing specific binding. It should be apparent to those skilled in the art that additional charges can be added to the zwitterionic imaging dyes, if needed, to balance overall surface charge to zero.

In certain embodiments, the imaging dye can include a reactive linking group. Such reactive linking groups are typically an activated derivative of a carboxylic acid, such as an n-hydroxysuccinimide (NHS) ester, a sulfo-NHS ester, a pentafluorophenyl (PFP) ester, a hydroxybenzotriazole (HOBt) ester, a hydroxyazabenzotriazole (HOAt) ester, a tetrafluorophenyl (TFP) ester, an acid anhydride, an acid azide or an acid halide. Such reactive linking groups can be bound or substituted onto the chelator at any suitable structural location as would be understood by one of ordinary skill in the synthesis of such compounds. Reactive linking groups also include, but are not limited to, alkynes, azides, maleimides, thiols, amines, alkohols, phenols, carbonyls, phosphanes, alkenes and tetrazines.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish, parasites, microbes, and the like.

As used herein, the term “administration” or “administering” of the subject compound refers to providing a compound of the invention and/or prodrugs thereof to a subject in need of diagnosis or treatment.

As used herein, the term “carrier” refers to chemical compounds or agents that facilitate the incorporation of a compound described herein into cells or tissues.

As used herein, the term “acceptable” with respect to a formulation, composition or ingredient, as used herein, means having no persistent detrimental effect on the general health of the subject being treated.

As used herein, the term “diluent” refers to chemical compounds that are used to dilute a compound described herein prior to delivery. Diluents can also be used to stabilize compounds described herein.

As used herein, the term “contacting” refers to the bringing together of substances in physical contact such that the substances can interact with each other. For example, when an imaging agent is “contacted” with tissue or cells, the tissue or cells can interact with the imaging agent, for example, allowing the possibility of binding interactions between the agent and molecular components of the tissue or cells. “Contacting” is meant to include the administration of a substance such as an imaging agent of the invention to an organism. Administration can be, for example, oral or parenteral.

As used herein, the term “ionic group” refers to a moiety comprising one or more charged substituents. The “charged substituent” is a functional group that is generally anionic or cationic when in substantially neutral aqueous conditions (e.g. a pH of about 6.5 to 8.0 or about physiological pH (7.4)). As recited above, examples of charged anionic substituents include anions of inorganic and organic acids such as sulfonate (—SO31-), sulfinate, carboxylate, phosphinate, phosphonate, phosphate, and esters (such as alkyl esters) thereof. In some embodiments, the charged substituent is sulfonate. Examples of charged cationic substituents include quaternary amines (—NR3+), where R is independently selected from C1-6 alkyl, aryl, and arylalkyl. Other charged cationic substituents include protonated primary, secondary, and tertiary amines, and well as guanidinium. In some embodiments, the charged substituent is N(CH3)3+.

As used herein, the phrase “non-ionic oligomeric or polymeric solubilizing groups” refers to soluble polymers such as, for example, polyethylene glycol, polypropylene glycol, polyethylene oxide and propylene oxide copolymer, a carbohydrate, a dextran, polyacrylamide, and the like. The solubilizing group can be attached by any desired mode. The point of attachment can be, e.g., a carbon-carbon bond, a carbon-oxygen bond, or a nitrogen-carbon bond. The attachment group can be, e.g., an ester group, a carbonate group, a ether group, a sulfide group, an amino group, an alkylene group, an amide group, a carbonyl group, or a phosphate group.

Some examples of solubilizing groups include polyethylene glycols, such as (CH2CH2O)a-H, —OC(═O)O(CH2CH2O)aH, —OC(═O)O(CH2CH2O)aCH3, —O(CH2CH2O)aCH3, and —S(CH2CH2O)2CH3, “a” being an integer between about 2 and about 25O. In some embodiments, “a” is 4 to 12 or 5 to 10. In further embodiments, “a” is 6, 7, or 8. Other examples of solubilizing groups include dextrans such as —OC(═O)O(dextran).

The solubilizing moiety can have an absolute molecular weight of from about 500 amu to about 100,000 amu, e.g., from about 1,000 amu to about 50,000 amu or from about 1,500 to about 25,000 amu.

Further examples of solubilizing groups include: —(CH2)c-(OCH2CH2)d-ORa, wherein “c” is 0 to 6, “d” is 1 to 200, and Ra is H or C1-6 alkyl. In some embodiments, “c” is 1 to 4, “d” is 1 to 10, and Ra is H. In some embodiments, “d” is 6 or 7.

See WO 2008/017074, U.S. Ser. No. 12/376,243 (filed Feb. 3, 2009), and U.S. Ser. No. 12/376,225 (filed Feb. 3, 2009), each of which is incorporated herein by reference in its entirety, for a further description of suitable non-ionic oligomeric or polymeric solubilizing groups, and method for incorporating them into dyes.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Compounds of the invention can also include all isotopes of atoms occurring in the intermediates or final compounds. Isotopes include those atoms having the same atomic number but different mass numbers. For example, isotopes of hydrogen include tritium and deuterium.

The chemical substances represented herein by name, chemical formula, or structure are meant to include all stereoisomers, geometric isomers, tautomers, resonance structures, and isotopes of the same, unless otherwise specified.

The chemical substances described herein may be charged or include substituents with formal charges. When such chemical substances are represented as charged, it is understood that, unless otherwise specified, the charges are generally countered with an appropriate counterion. For example, chemical substances or functional groups having a charge of −1 are understood to be countered with an ion have a +1 charge. Suitable counterions with +1 charge include Na+, K+, tetraalkylammonium ions, and the like. Conversely chemical substances or functional groups having a charge of +1 are understood to be countered with an ion having a-1 charge. Suitable counterions with −1 charge include F—, Cl—, Br—, I—, sulfate, phosphate, perchlorate, acetate, trifluoroacetate, maleate, fumarate, mesylate, lactate, pyruvate, laevulinate, gluconate and the like.

### Imaging Dyes

In at least one aspect, the invention provides an imaging agent dye comprising a charge-balanced imaging agent conjugated to targeting vector. In certain embodiments, the charge-balanced imaging agent can be ZW-800-1, or ZW-830-1, or ZW-700-1-Forte, which can have the formulas shown herein and in the Figures.

The imaging agent of the invention is particularly advantageous because their behavior in vivo is believed to contribute to superior optical imaging properties and superior stability. More specifically, the charge-balancing is believed to impart good biodistribution and clearance properties to the agents, and reduce undesirable non-specific binding while the inclusion of the targeting ligand increases the time of circulation and contact time with the target. These in vivo properties help improve the target-to-background ratio of imaged tissues, leading to higher resolution imaging.

In at least one aspect, the invention provides, an imaging agent can include, a ZW-800-1 dye, a ZW-830-1 dye, or a ZW-700-1-Forte dye. In accordance with at least one aspect of the invention, imaging dye can be conjugated to a targeting ligand or targeting vector, discussed in the following section.

In certain embodiments of the invention, the imaging dye according to the invention has the following structure (or is a salt, solvate, or hydrate thereof, or is a dimer thereof):

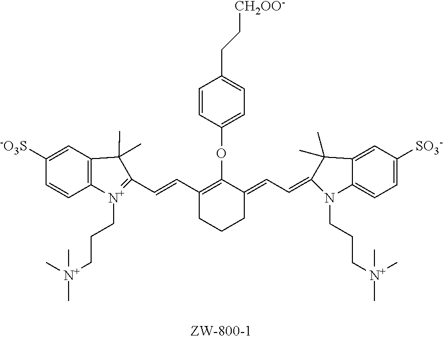

In certain embodiments of the invention, the imaging dye according to the invention has the following structure (or is a salt, solvate, or hydrate thereof, or is a dimer thereof):

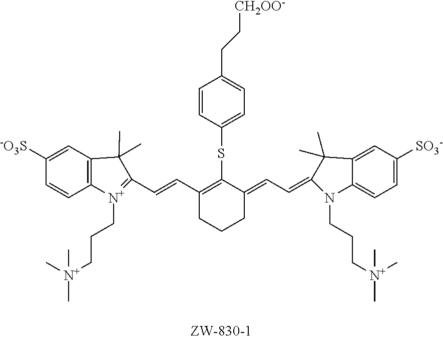

In certain embodiments of the invention, the imaging dye according to the invention has the following structure (or is a salt, solvate, or hydrate thereof, or is a dimer thereof):

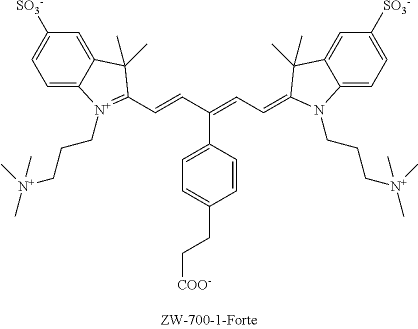

### Targeting Ligands or Vectors

In at least one aspect of the invention provides, the imaging agent includes an embodiment of one of the aforementioned imaging dyes conjugated to a targeting ligand or targeting vector. In certain embodiments, the targeting vector (or targeting ligands) can be cRGD, PSMA binding vector, such as dPSMA-617 or KUE, a FAP binding vector such as NH₂-FAPI-74, a bombesin receptor binding vector, or a somatostatin receptor binding vector, or their corresponding homo- or hetero-dimers. In certain embodiments, the charge-balanced imaging agent is conjugated to the targeting vector via a direct bond, or via a linking group, for example as shown in the formulas described herein, and in the Figures.

In certain embodiments, one or more of the imaging agent conjugates in the combination includes cRGD as the targeting ligand. The imaging agent conjugates in the combination include one or more of cRGD-ZW800-1, cRGD-ZW700-1 Forte, or cRGD-ZW830-1.

In certain embodiments of the invention, the targeting ligand according to the invention can be a cyclic-RGD having the following structure:

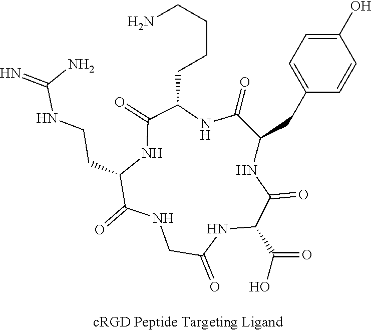 [SMILES: N=C(N)NCC[C@@H]1NC(=O)[C@H](CCCCN)NC(=O)[C@@H](Cc2ccc(O)cc2)NC(=O)[C@H](C(=O)O)NC(=O)CNC1=O]

Provided below are examples for cRGD conjugates for embodiments of the imaging agents, including cRGD-ZW800-1, cRGD-ZW830-1, or cRGD-ZW700-1-Forte. The targeting ligand can be conjugated to the imaging dye by an optional linking group or via direct bond. As shown herein, the imaging targeting ligand is conjugated to the imaging dye via an amine linkage. Nevertheless, other conjugating linkages can be used as determined by one of skill in the art. Other cRGD conjugates can be prepared without deviating from the essence of the invention.

In certain embodiments of the invention, the targeting ligand conjugated to the imaging dye can have the following formula:

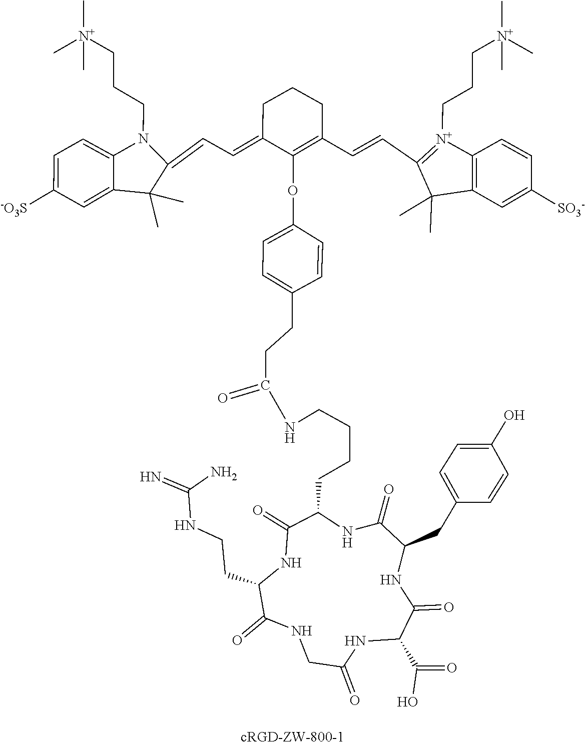

In certain embodiments of the invention, the targeting ligand conjugated to the imaging dye can have the following formula:

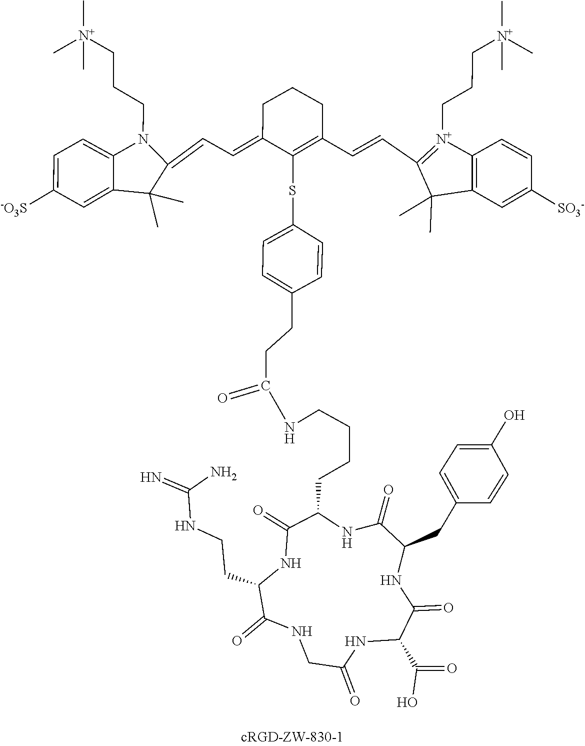

In certain embodiments of the invention, the targeting ligand conjugated to the imaging dye can have the following formula:

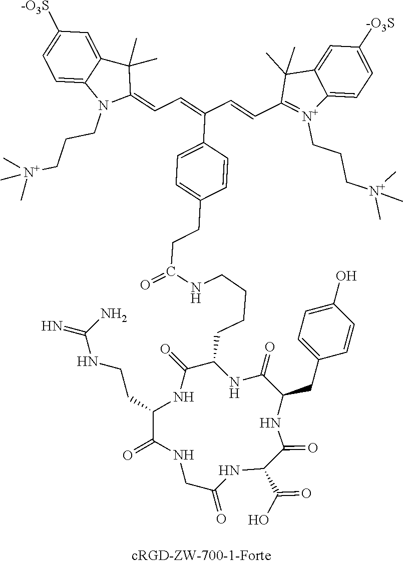

Integrins are a family of cell adhesion receptors that provide for cell motility and invasion. The integrin family in humans comprise 18 a and B subunits which assemble into different functional heterodimers. Some integrins, act as mediators of angiogenesis in solid tumors. Integrin expression is important for tumor progression and metastasis by promoting tumor cell migration, invasion, proliferation, and survival. In addition to cancer, integrins are also highly expressed during many normal and abnormal processes, such as fibrosis, wound healing, and inflammation. Integrin αvβ3 expression has been established as a surrogate marker of angiogenic activity. Similarly, Integrin αvβ6 is a subtype of the integrin family that is expressed exclusively on epithelial cells. αvβ6 is usually expressed at low or undetectable levels in normal adult tissues but can be highly upregulated during pathological and physiological processes such as wound healing, fibrosis, inflammation, and cancer. RGD mimetics have been used in some studies as a targeting moiety to deliver integrin antagonists to a variety of cell types. However, continuous infusion of RGD peptides have been found to stimulate tumor growth (See, Reynolds, L. E. et al. Enhanced pathological angiogenesis in mice lacking β3 integrin or β3 and β5 integrins. Nature Med. 8, 27-34 (2002)). In addition, many RGD bound agents have expensive, complex, time-consuming and low-yield synthetic procedures. Similarly, with regard to imaging, degradation of agents accumulation of RGD bound agents in the bladder have been found to impair some imaging methods. Given their stability and clearance rates, the conjugates of the invention have the potential to provide a long-lasting visual identification and imaging of tumors which overexpress integrins while avoiding the setbacks of prolonged systemic administration of RGD peptide ligands.

As such, in a particular aspect, the charge-balanced imaging agent may be used to image or identify (for example, during surgery) integrin overproduction in the cells of a subject, for example when the imaging dye compound is conjugated to cRGD, e.g., as shown in the above structures.

In certain embodiments of the invention, the targeting ligand according to the invention can be a prostate specific membrane antigen (PSMA) binding vector. For example, dPSMA-617 or Vipivotide tetraxetan. In certain embodiments, the PSMA binding vector can be F a targeting vector based on (((S)-5-((S)-2-((1r,4S)-4-(aminomethyl) cyclohexane-1-carboxamido)-3-(naphthalen-2-yl) propanamido)-1-carboxypentyl) carbamoyl)-L-glutamic acid, which is a derivative of PSMA-617 or Vipivotide tetraxetan. This PSMA-targeting vector is called dPSMA-617 (for “derivative of PSMA-617) in this disclosuredPSMA-617. In certain such embodiments, the imaging agent conjugates in the combination include one or more of dPSMA-617-ZW800-1, dPSMA-617-ZW700-1 Forte, or dPSMA-617-ZW830-1.

In certain embodiments of the invention, the targeting ligand according to the invention can be dPSMA-617 having the following structure:

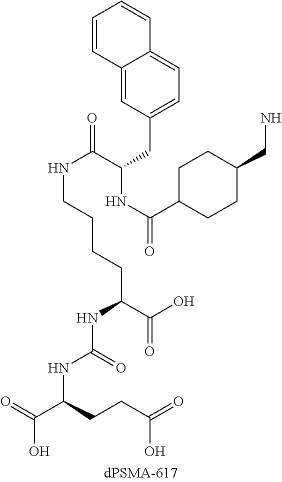

Provided below are examples for dPSMA-617 conjugates for embodiments of the imaging agents, including dPSMA-617-ZW800-1, dPSMA-617-ZW830-1, or dPSMA-617-ZW700-1-Forte. The targeting ligand can be conjugated to the imaging dye by an optional linking group or via direct bond. As shown herein, the imaging targeting ligand is conjugated to the imaging dye via an amine linkage. Nevertheless, other conjugating linkages can be used as determined by one of skill in the art. Other dPSMA-617 conjugates can be prepared without deviating from the essence of the invention.

In certain embodiments of the invention, the targeting ligand conjugated to the imaging dye can have the following formula:

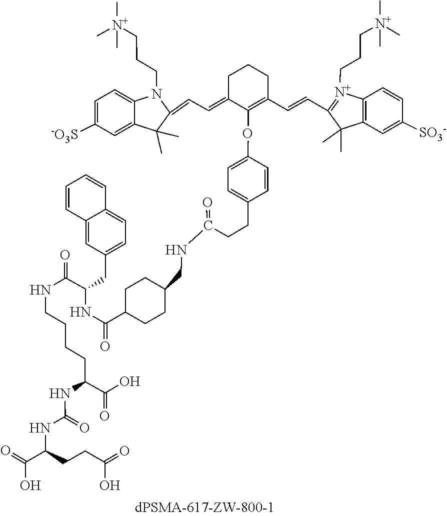

In certain embodiments of the invention, the targeting ligand conjugated to the imaging dye can have the following formula:

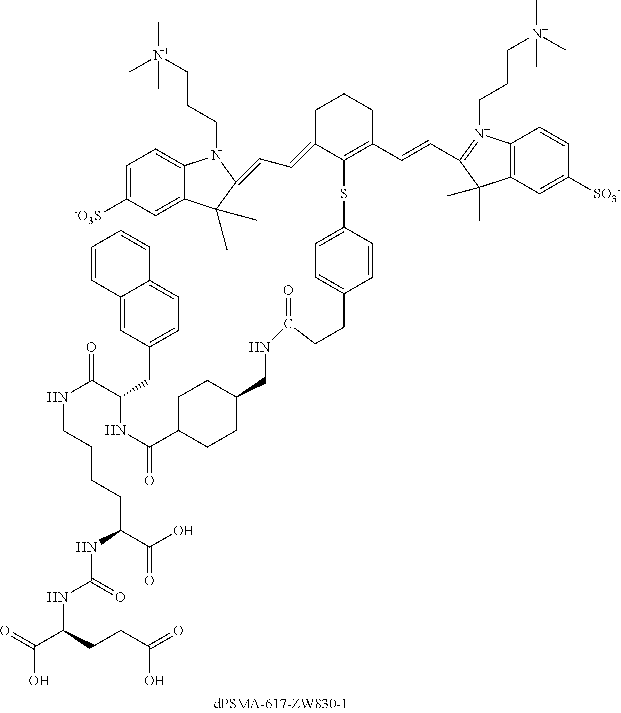

In certain embodiments of the invention, the targeting ligand conjugated to the imaging dye can have the following formula:

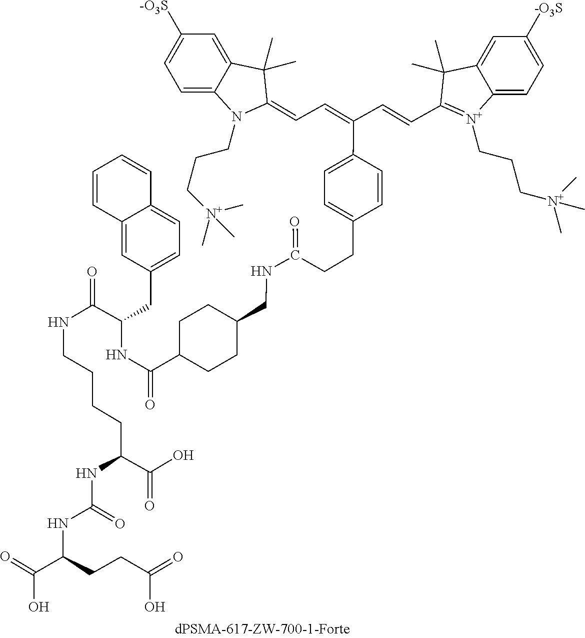

In certain embodiments of the invention, the PSMA-targeting vector is (((S)-5-amino-1-carboxypentyl) carbamoyl)-L-glutamic acid (KUE). In certain such embodiments, the imaging agent conjugates in the combination include one or more of KUE-ZW800-1, KUE-ZW700-1 Forte, or KUE-ZW830-1. The targeting ligand KUE can have the following structure:

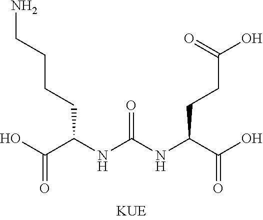 [SMILES: NCCCC[C@H](NC(=O)N[C@@H](CCC(=O)O)C(=O)O)C(=O)O]

Provided below are examples for KUE conjugates for embodiments of the imaging agents, including KUE-ZW800-1, KUE-ZW830-1, or KUE-ZW700-1-Forte. The targeting ligand can be conjugated to the imaging dye by an optional linking group or via direct bond. As shown herein, the imaging targeting ligand is conjugated to the imaging dye via linking group (L). Nevertheless, other conjugating linkages can be used as determined by one of skill in the art. Other KUE conjugates can be prepared without deviating from the essence of the invention.

In certain embodiments of the invention, the targeting ligand conjugated to the imaging dye can have the following formula:

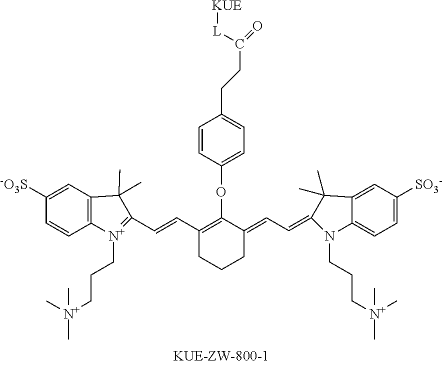

In certain embodiments of the invention, the targeting ligand conjugated to the imaging dye can have the following formula:

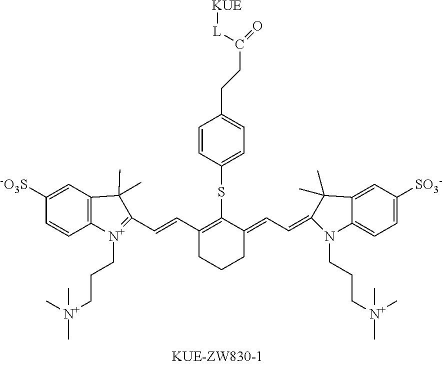

In certain embodiments of the invention, the targeting ligand conjugated to the imaging dye can have the following formula:

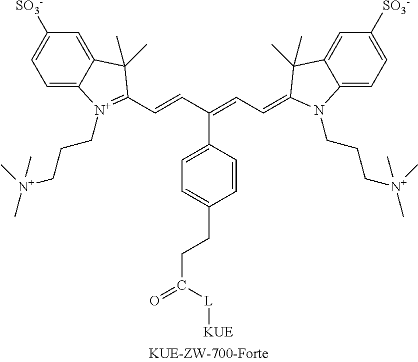

In certain embodiments of the invention, the targeting ligand according to the invention can be a FAP binding vector or a FAP-targeting molecule (FAP-inhibitor or FAPI). In certain such embodiments, the imaging agent conjugates in the combination include one or more of FAP-ZW800-1, FAP-ZW700-1 Forte, or FAP-ZW830-1.

In certain embodiments, the FAPI-targeting ligand includes NH2-FAPI-74. Specific details regarding NH2-FAPI-74 are described in: Linder et al., “Radioligands Targeting Fibroblast Activation Protein (FAP),” the entirety of which is incorporated herein by reference.

In general, NH2-FAPI-74 has the following structure:

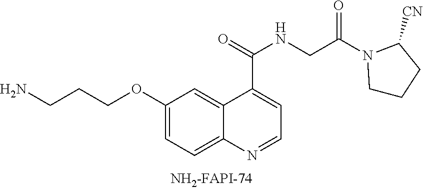 [SMILES: N#C[C@@H]1CCCN1C(=O)CNC(=O)c1ccnc2ccc(OCCCN)cc12]

Provided below are examples for FAP conjugates for embodiments of the imaging agents, including FAP-ZW800-1, FAP-ZW830-1, or FAP-ZW700-1-Forte. The targeting ligand can be conjugated to the imaging dye by an optional linking group or via direct bond. As shown herein, the imaging targeting ligand is conjugated to the imaging dye via linking group (L). Nevertheless, other conjugating linkages can be used as determined by one of skill in the art. Other FAP conjugates can be prepared without deviating from the essence of the invention.

In certain embodiments of the invention, the targeting ligand conjugated to the imaging dye can have the following formula:

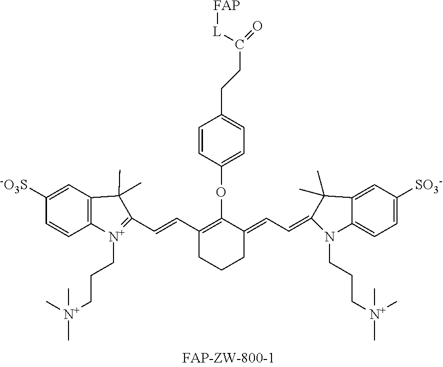

In certain embodiments of the invention, the targeting ligand conjugated to the imaging dye can have the following formula:

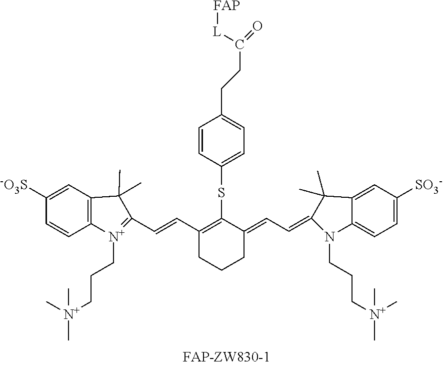

In certain embodiments of the invention, the targeting ligand conjugated to the imaging dye can have the following formula:

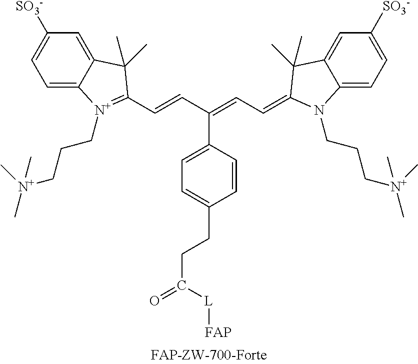

In certain embodiments of the invention, the targeting ligand according to the invention can be a bombesin receptor binding vector. In certain such embodiments, the imaging agent conjugates in the combination include one or more of BOMBESIN-ZW800-1, BOMBESIN-ZW700-1 Forte, or BOMBESIN-ZW830-1.

In general, the structure of bombesin (SEQ ID No.: 6) is:

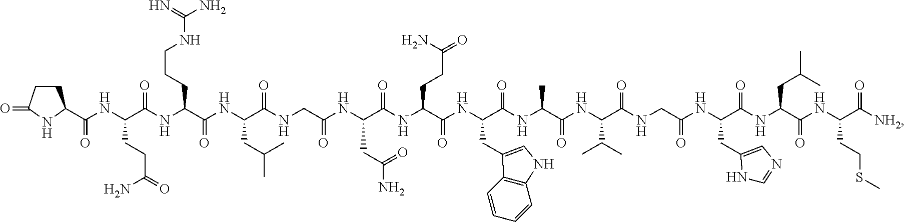 [SMILES: CSCC[C@H](NC(=O)[C@H](CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@H](CC(N)=O)NC(=O)CNC(=O)[C@H](CC(C)C)NC(=O)[C@H](CCCNC(=N)N)NC(=O)[C@H](CCC(N)=O)NC(=O)[C@@H]1CCC(=O)N1)C(C)C)C(N)=O]

or a derivative thereof.

Provided below are examples for BOMBESIN conjugates for embodiments of the imaging agents, including BOMBESIN-ZW800-1, BOMBESIN-ZW830-1, or BOMBESIN-ZW700-1-Forte. The targeting ligand can be conjugated to the imaging dye by an optional linking group or via direct bond. As shown herein, the imaging targeting ligand is conjugated to the imaging dye via linking group (L). Nevertheless, other conjugating linkages can be used as determined by one of skill in the art. Other bombesin conjugates can be prepared without deviating from the essence of the invention.

In certain embodiments of the invention, the targeting ligand conjugated to the imaging dye can have the following formula:

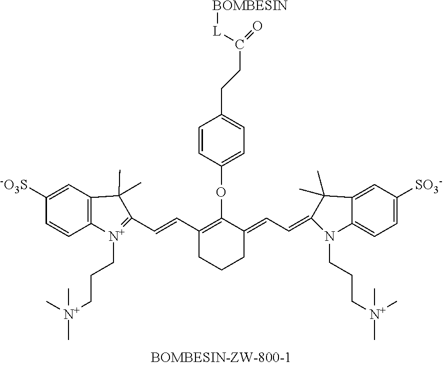

In certain embodiments of the invention, the targeting ligand conjugated to the imaging dye can have the following formula:

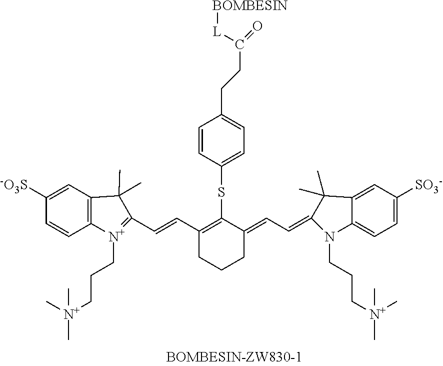

In certain embodiments of the invention, the targeting ligand conjugated to the imaging dye can have the following formula:

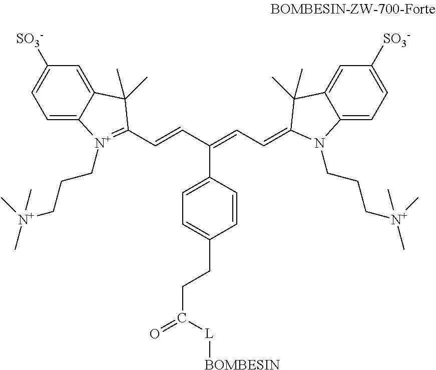

In certain embodiments of the invention, the targeting ligand according to the invention can be a somatostatin receptor binding vector. In certain embodiments, the somatostatin receptor binding vector can be octreotide. In certain such embodiments, the imaging agent conjugates in the combination include one or more of OCTREOTIDE-ZW800-1, OCTREOTIDE-ZW700-1 Forte, or OCTREOTIDE-ZW830-1.

In general, the structure of octreotide (SEQ ID No.: 5) is:

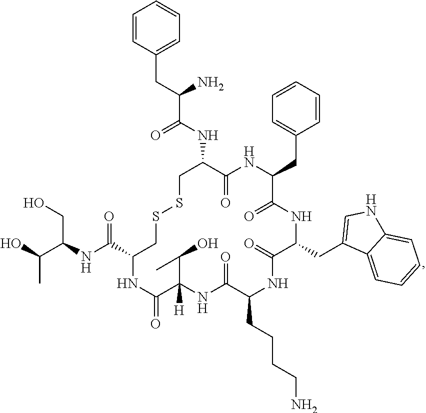 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O]

or a derivative thereof.

Provided below are examples for octreotideconjugates for embodiments of the imaging agents, including OCTREOTIDE-ZW800-1, OCTREOTIDE-ZW830-1, or OCTREOTIDE-ZW700-1-Forte. The targeting ligand can be conjugated to the imaging dye by an optional linking group or via direct bond. As shown herein, the imaging targeting ligand is conjugated to the imaging dye via linking group (L). Nevertheless, other conjugating linkages can be used as determined by one of skill in the art. Other OCTREOTIDE conjugates can be prepared without deviating from the essence of the invention.

In certain embodiments of the invention, the targeting ligand conjugated to the imaging dye can have the following formula:

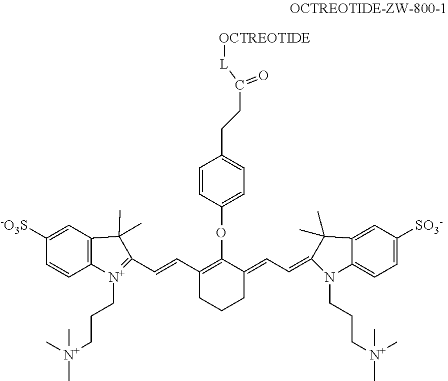

In certain embodiments of the invention, the targeting ligand conjugated to the imaging dye can have the following formula:

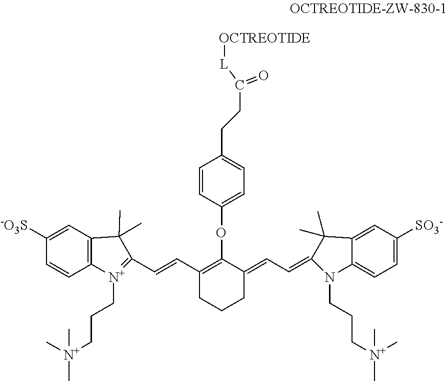

In certain embodiments of the invention, the targeting ligand conjugated to the imaging dye can have the following formula:

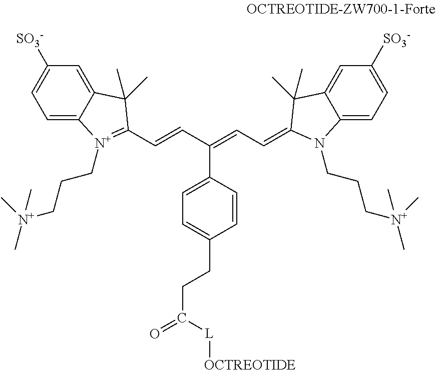

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

Accordingly, the invention provides an imaging agent dye comprising a charge-balanced imaging agent conjugated to a targeting vector. In certain embodiments of the imaging agent dye according to the invention, the charge-balanced imaging agent can be ZW-800-1, ZW-830-1, or ZW-700-1-Forte and the targeting vector can be a PSMA ligand, for example, KUE or dPSMA-617, a FAP ligand, a bombesin receptor ligand, or a somatostatin receptor ligand, as shown and described. The charge-balanced imaging agent can be conjugated to the targeting vector via a direct bond, or via a linking group.

Any suitable combination of charge-balanced imaging agent and targeting ligand can be used, for example, the three charge-balanced imaging agents discussed herein, e.g., ZW-800-1, or ZW-830-1, or ZW-700-1-Forte can be used with any one of the targeting vectors discussed herein, e.g., cRGD, PSMA binding vector, such as dPSMA-617 or KUE, a FAP binding vector, a bombesin receptor binding vector, or a somatostatin receptor binding vector.

In each combination, the respective targeting ligand or vector can be covalently attached to a respective reactive linking group of a respective dye compound of the invention through standard coupling procedures. For example, the carboxyl or activated carboxyl group of the reactive linking group can react with a nucleophilic functionality on the targeting ligand, such as an amine or alkoxy derivative, to form an amide or ester linkage. Additional details for the conjugation of dyes can be found in WO 2008/017074 and in Frangioni et al. Molecular Imaging, Vol. 1(4), 354-364 (2002), each of which is incorporated herein by reference in its entirety.

In certain embodiments, the targeting vector can be a targeting ligand and can be covalently attached to a reactive group of the dye compound, or through an optional linking group (L), through standard coupling procedures. For example, the carboxyl or activated carboxyl group of the reactive linking group can react with a nucleophilic functionality on the targeting ligand, such as an amine or alkoxy derivative, to form an amide or ester linkage. Additional details for the conjugation of dyes can be found in WO 2008/017074 and in Frangioni et al. Molecular Imaging, Vol. 1(4), 354-364 (2002), each of which is incorporated herein by reference in its entirety.

As used herein, “linking group” refers to any molecular entity having a molecular weight from about 50 to about 500 Da that is capable of conjugating with a targeting ligand (TL). In particular, the linking group includes at least one reactive group selected from a carboxylic acid group or anhydride or ester thereof, as well as an isothiocyanate group. In some embodiments, the linking group contains a carboxylic acid group. In some embodiments, the linking group is a PEG moiety or a straight or branched chain hydrocarbon moiety having between 2 and 12 carbon atoms. In some embodiments, the linking group includes a branching point, a reactive linking group, or other reactive group which can be used to further functionalize the imaging agent, for example, for inclusion of a radioisotope.

In certain embodiments, the targeting ligand can further include a molecular scaffold moiety to which the binding moiety and other groups can attach. For example, the molecule scaffold can bear one or more of the following: (1) a moiety designed to react with the reactive linking group of the dye to form a covalent bond, (2) a charge balancing moiety, such as any of the ionic groups described herein, and (3) a moiety that binds to the biological target. An example of a molecular scaffold is an adamantane derivative, such as described in U.S. Pat. App. Pub. No. 2006/0063834, which is incorporated herein by reference in its entirety, and illustrates the preparation of a targeting ligand that incorporates an adamantane scaffold. Specifically, the adamantane core holds (1) an amino group capable of reacting with the dye compounds, (2) a charge-balancing moiety that will neutralize a negative charge on the dye molecule, and (3) two moieties that bind to the biological target PSMA. For a description of moieties that bind to PSMA, see, Humblet, V. et al. Mol. Imaging, 2005, 4:448-62; Misra P. et al. J. Nucl. Med. 2007, 48:1379-89; Chen, Y., et al. J. Med. Chem, 2008, 51:7933-43; Chandran, S. S., et al. Cancer Biol. Ther., 2008, 7:974-82; Banerjee, S. R., J. Med. Chem. 2008, 51:4504-17; Mease, R. C., et al. Clin. Cancer Res., 2008, 14:3036-43; Foss, C. A. et al. Clin. Cancer. Res., 2005, 11:4022-8, each of which is incorporated herein by reference in its entirety.

In certain embodiments, ZW-800-1, ZW-830-1, ZW-700-1-Forte, targeting ligands, and imaging agents can be isolated as salts, acids, bases, or combinations thereof. For example, dyes, conjugates, and imaging agents having multiple charged substituents can be isolated by introducing counterions and/or protons sufficient to counter the charges of the various substituents normally present in neutral pH so that the dye, conjugate, or imaging agent can be isolated, for example, as a solid substance.

In certain embodiments, the imaging agent further comprises a PEG-moiety. Such moiety can be bound to the conjugate at any suitable structural location as would be understood by one of ordinary skill in the synthesis of such compounds. In certain embodiments, the agent further comprises a PEG-moiety to alter the circulation time in blood. Such moiety can be bound to the conjugate at any suitable structural location as would be understood by one of ordinary skill in the synthesis of such compounds. In addition, in certain embodiments, the PEG-moiety can be included as the optional linking group between ZW-800-1, ZW-830-1, ZW-700-1-Forte and the targeting ligand using methods known to one of ordinary skill in the art.

In certain embodiments, the imaging agent can include a radioisotope for either single-photon emission computed tomography (SPECT) or positron emission tomography (PET). Such radioisotope can be bound or further conjugated to the conjugate at any suitable structural location as would be understood by one of ordinary skill in the synthesis of such compounds.

In certain embodiments, the imaging agent can comprise a reactive linking group. Such reactive linking groups are typically an activated derivative of a carboxylic acid, such as an n-hydroxysuccinimide (NHS) ester, a sulfo-NHS ester, a pentafluorophenyl (PFP) ester, a hydroxybenzotriazole (HOBt) ester, a hydroxyazabenzotriazole (HOAt) ester, a tetrafluorophenyl (TFP) ester, an acid anhydride, an acid azide or an acid halide. Such reactive linking groups can be bound or substituted onto the chelator at any suitable structural location as would be understood by one of ordinary skill in the synthesis of such compounds. Reactive linking groups also include, but are not limited to, alkynes, azides, maleimides, thiols, amines, alkohols, phenols, carbonyls, phosphanes, alkenes and tetrazines.

### Imaging Methods, Methods of Identifying and Imaging Tumors, and Methods of Treating Cancer

Embodiments of the imaging agents, including dye compounds conjugated to antigen specific targeting vectors can be particularly advantageous because their behavior in vivo can contribute to superior optical imaging properties, for example, by significantly increasing the target-to-background ratio of imaged tissues, leading to higher resolution imaging, and ultimately providing for better recognition of malignant tissue for resection and margin assessment and improved visualization during minimal invasive laparoscopic surgery, for example. Performing complete and accurate resections of cancerous tissues during surgery is critical, thus higher target-to-background ratios observed by using embodiments of the imaging agents in conjunction with one or more embodiments of the inventive methods described herein, provides clinicians with greater visualization to ensure resections are complete.

Moreover, embodiments of imaging agents according to the invention include a labile linking bond that when broken renders the fluorophore non-fluorescent in the NIR spectrum and separates the targeting ligand from the fluorophore. If the targeting ligand binds to a cell and is internalized within lysosomes or other acidic intracellular compartments, the imaging dye is stabilized. The NIR fluorophores of this invention are highly stable in acidic environments and thus display maximum NIR fluorescence. On the contrary, non-bound targeted fluorophore is degraded over time in blood, thus lowering the background significantly. The combination of increased NIR fluorophore stability after targeting and internalization and decreased stability in blood results in a counterintuitive result that forms the basis of this invention. In this disclosure, it has been found that TBR actually increases over time rather than decreases over time and that surprisingly high TBR can be seen 2-24 hours after administration. In certain embodiments, peak TBR can be seen 4-10 hours after intravenous injection, 12-24 hours after intravenous injection, 15-18 hours after intravenous injection, or even 18-24 hours after intravenous injection. On the contrary, currently known targeted NIR fluorophores described in literature display peak TBR at short time points (2-6 hours) after injection, with a steady decrease thereafter. In certain embodiments, the labile linking bond can be a labile —O— ether bond, such as shown in ZW-800 conjugates. In certain embodiments, the labile linking bond can be a —S— bond, such as shown in the ZW-830 conjugates. In still other embodiments, the labile linking bond can be an —NH— bond wherein the targeting ligand is bound to a fluorophore core such as the core shown below:

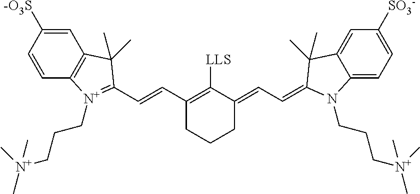

wherein LLS represents the labile linker bond and coordinated targeting ligand, optionally including an additional linker between the labile linker bond and the targeting ligand.

The labile bond allows for stabilization of the imaging dye after contact with the target cell and internalization into intracellular organelles. Said differently, the imaging dye will be unstable in plasma because of the labile bond but, the imaging dye has increased stability when in the acid environment of lysosomes, e.g., after internalization. Thus, internalized dye is bright but non-internalized (i.e., non-binding dye) gets degraded and fluorescence of the non-internalized dye is stopped when the targeting ligand breaks from the imaging dye at the labile linking bond. This results in higher TBR because only the internalized dye fluoresces bright, while non-internalized dye does not fluoresce.

In at least one aspect, the invention provides, a method of imaging cells, e.g., tumor cells in a subject, where the cells can be tumor cells, inflammatory cells, or cells undergoing angiogenesis, and the subject can be a human subject, and further, the cells can be imaged in vivo.

The methods of imaging tissue or cells include the following basic steps:
(a) contacting the tissue or cells with an imaging agent comprising a conjugate of a dye, the conjugate comprising a PSMA, FAP, bombesin receptor, or somatatostatin receptor targeting ligand bound to the dye;
(b) irradiating the tissue or cells at a wavelength absorbed by the conjugate; and
(c) detecting an optical signal from the irradiated tissue or cells.

In certain embodiments, the method according to the invention can include the following steps:
a) administering an imaging effective amount of an imaging agent according to any one or more embodiments of the invention described herein to the subject;
b) 2-4 hours after administration of the imaging agent, irradiating a region in the subject in which tumor cells are expected to be found with at a wavelength absorbed by the imaging agent; and
c) detecting a signal from the imaging agent, thereby imaging the tumor cells, where the target-to-background ratio 2-4 hours after administration is from about 2.75 to about 15.

In certain embodiments, the imaging agents used in accordance with at least one embodiment of the method can achieve an TBR of at least target-to-background ratio about 3 to about 12.5. In further embodiments of the method, the imaging agents can achieve an TBR of at least about 4 to about 10. In further embodiments of the method, the imaging agents can achieve an TBR of at least target-to-background ratio greater than about 4.2, or more particularly, about 6.2, or still more particularly, the imaging agents can achieve an TBR of at least target-to-background ratio about 7.6 2-24 hours after administration, reaching peak fluorescence at a time of about 18-24 hours after administration. In certain embodiments, the target-to-background ratios discussed above are present 4-10 hours after administration. In certain embodiments, the target-to-background ratios discussed above are present 12-24 hours after administration. In certain embodiments, the target-to-background ratios discussed above are present 15-18 hours after administration. In certain embodiments, the target-to-background ratios discussed above are present 18-24 hours after administration.

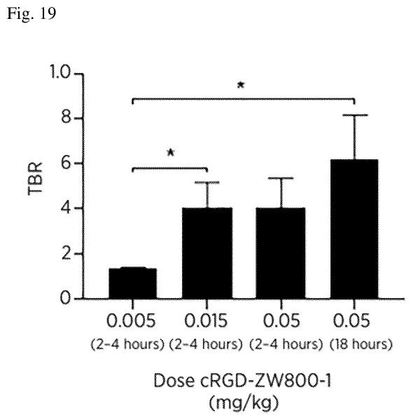

In certain embodiments, the target-to-background ratio 2-4 hours after administration can be a two-fold or more increase than a target-to-background ratio 2-4 hours after administration of a fluorophore without an embodiment of a targeting ligand as described herein. See, for example, FIG. 19, which demonstrates the calculated ex vivo TBR for colon tumors at the different dose levels and injection time windows, where the stars represent a significant increase in ex vivo TBR. Further discussion of TBR, for example as it relates to cRGD-ZW800-1, is provided in Kim S. de Valk et al., “First-in-Human Assessment of cRGD-ZW800-1, a Zwitterionic, Integrin-Targeted, Near-Infrared Fluorescent Peptide in Colon Carcinoma,” Clinical Cancer Research|Precision Medicine and Imaging, Apr. 28, 2020, the entirety of which is incorporated herein by reference.

In at least one aspect, the invention provides a method of imaging cells, and further, a method of treating cancer in a subject.

In certain embodiments of the method according to the invention, the method of treating cancer can further include, imaging the cells, which can include the following steps:
a) administering an imaging effective amount of an imaging agent according to any one or more embodiments of the invention described herein (e.g., an imaging agent comprising a conjugate of a dye, the conjugate comprising a PSMA, FAP, bombesin receptor, or somatostatin receptor targeting ligand bound to the dye) to a subject; and
b) irradiating the cells, tissues or organs of a subject suspected of being cancerous at a wavelength absorbed by the imaging agent. In certain embodiments, the imaging agent has peak absorbance at about 600 nm to 850 nm.

The method for treating cancer, can further include the following steps,
c) diagnosing the cancer in the cells tissues, or organs of the subject by detecting a signal from the imaging agent; and
d) administering a chemotherapeutic treatment, a radiotherapeutic treatment, or a surgical treatment to the subject to treat the cancer.

In certain embodiments, the method is suitable for imaging of abnormal, but not malignant, tissue, such as defects of the musculoskeletal system using FAP as a targeting ligand, vascular system using cRGD as a targeting ligand, melanoma using LyP-1 peptide as a targeting ligand, breast cancer using K237 peptide and/or mUNO as a targeting ligand, Lung tumor, breast tumor, colon tumor using IL4RPep-1 as a targeting ligand.

In certain embodiments, the method is suitable for imaging of cancer, tumor or neoplasm. In a further embodiment, the cancer is selected from eye or ocular cancer, rectal cancer, colon cancer, cervical cancer, prostate cancer, breast cancer and bladder cancer, oral cancer, benign and malignant tumors, stomach cancer, liver cancer, pancreatic cancer, lung cancer, corpus uteri, ovary cancer, prostate cancer, testicular cancer, renal cancer, brain/ns cancer (e.g., gliomas), throat cancer, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, neurofibromatosis, tuberous sclerosis, hemangiomas, and lymphangiogenesis.

In certain embodiments, the cancer cells are adult solid tumor cells or pediatric solid tumor cells. Non-limiting examples of such cells include melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

In particular embodiments, the cancer cells are prostate cancer cells.

In certain embodiments, the biological sample is part or all of a subject. In some embodiments, the biological sample is obtained from a subject.

In particular embodiments, the method includes the steps of (a) contacting the biological sample with a combination of the imaging agent conjugates described above, wherein each of the imaging agent conjugates in the combination comprises a targeting ligand linked to a charge-balanced imaging agent. The charge-balanced imaging agent is capable of being detected by one or more conventional scanning methods.

In certain embodiments, the compound is administered by parenteral, intranasal, sublingual, rectal, or transdermal delivery. In some such embodiments, the compound is administered intravenously. In some embodiments, the compound is administered intratumorally.

In particular embodiments, the cells are tumor cells. For example, and without limitation, the tumor cells are melanoma cells, sarcoma cells, musculoskeletal tumor cells, breast cancer tumor cells, renal cancer cells, rectal cancer tumor cells, bone metastases, neuroendocrine tumor cells, brain metastases, glioblastoma multiforme (GBM) cells, squamous cell carcinoma cells of the head and neck (SCCHN), or non-small cell lung cancer (NSCLC) cells. As such, the invention provides for a method of imaging such tumors in a subject.

In certain embodiments, the cells are associated with inflammation or angiogenesis. For example, and without limitation, the cells may be associated with inflammatory lesions, endometriosis, ischemic injury, cardiovascular disease, neurovascular disease, myocardial infarction, moyamoya disease, stroke, atherosclerosis, or rheumatoid arthritis.

In certain embodiments, and in at least one aspect, the invention provides, the method of imaging cells includes, contacting cells with an embodiment of an imaging agent according to any one or more embodiments of the invention described herein (e.g., having one of the formulas shown and described herein), irradiating the cells at a wavelength absorbed by the imaging agent, and detecting a signal from the imaging agent, thereby imaging the cells. The imaged cells can be tumor cells, inflammatory cells, or cells undergoing angiogenesis.

In certain embodiments of the method of imaging cells according to the invention, an embodiment of the imaging agent is administered to an organism comprising or suspected of comprising the cells. The imaging agent described herein is a substance that can be used to image tissues or cells, such as those of a living organism, for purposes of diagnosis, therapy, image-guided surgery, and the like. In some embodiments, the organism is a mammal, such as a human.

Certain embodiments of the imaging agent of the invention contains a dye that is capable of absorption of electromagnetic radiation, typically in the ultraviolet (UV), visible, or near infrared (NIR) range. The imaging agent can also be capable fluorescent emission, such as in the visible or NIR range. The optical signal detected from the dye or conjugate can be, for example, absorption or fluorescent emission. In some embodiments, fluorescent emission from the dye is the primary optical signal detected for imaging purposes. In some embodiments, the dye has a peak absorbance at about 525 nm to about 850 nm, at about 550 nm to about 825 nm, about 600 nm to about 825 nm, about 700 nm to about 825 nm, or at about 750 nm to about 825 nm. In some embodiments, the dye has a peak fluorescent emission at about 700 nm to 875 nm, about 725 to about 850 nm, about 750 to about 850 nm, or about 775 to about 850 nm. In particular embodiments, the conjugated imaging agent of the invention has a peak fluorescent emission at about 700 nm.

Certain embodiments of the imaging agent is generally “charge-balanced,” unless otherwise specified, which refers to having a net overall charge of zero, or close to zero, such as +1 or −1. Charge-balancing occurs when negatively charged substituents on the imaging agent are countered by the presence of an equal number, or close to an equal number, of positively charged substituents on the same molecule, and vice versa. In some embodiment, the net charge is 0 or +1. In some embodiments, the net charge is 0. In some embodiments, the net charge is +1. In further embodiments, the net charge is −1. The value “n” in the formulae provided herein represents net charge.

Certain embodiments of the imaging agent described herein generally has improved “target-to-background ratio” (TBR) compared to presently known fluorescent imaging agents. The improvement in TBR is believed to be a result of improved in vivo properties due to “charge-balancing.” TBR is a measure of the intensity of the fluorescent signal obtained from a target (peak signal) over the measure of the intensity of the fluorescent signal obtained nearby the target (background signal), the target being the tissues or cells targeted by the imaging agent. TBR measurements can be readily obtained through routine measurement procedures. For fluorescent imaging systems, and other optical-type systems, digital images recording optical signals of the target facilitate TBR measurement. Higher TBR values are more desirable, resulting in greater resolution of the imaged tissues. In some embodiments, the imaging agents achieve an TBR of at least about 1.1 (i.e., peak signal is at least 10% over background).

Certain embodiments of the imaging agent of the invention generally includes one or more ionic groups. In some embodiments, the imaging agents include two or more, three or more, four or more, or five or more ionic groups. Ionic groups serve to increase solubility of the generally hydrophobic dye portions of the imaging agent, thus, improving biodistribution. The ionic groups can be located on any portion of the imaging agent, such as the dye portion, the targeting ligand, or both.

The term “ionic group” refers to a moiety comprising one or more charged substituents. The “charged substituent” is a functional group that is generally anionic or cationic in substantially neutral aqueous conditions (e.g. a pH of about 6.5 to 8.0, or preferably about physiological pH (7.4)). Examples of charged anionic substituents include anions of inorganic and organic acids such as sulfonate (—SO₃¹⁻), sulfinate, carboxylate, phosphinate, phosphonate, phosphate, and esters (such as alkyl esters) thereof. In some embodiments, the charged substituent is sulfonate. Examples of charged cationic substituents include quaternary amines (—NR₃⁺), where R is independently selected from C₁₋₆ alkyl, aryl, and arylalkyl. Other charged cationic substituents include protonated primary, secondary, and tertiary amines, and well as guanidinium. In some embodiments, the charged substituent is —N(CH₃)₃⁺. Further examples of ionic groups are described infra.

Certain embodiments of the imaging agent of the invention generally has good solubility in substantially neutral aqueous media, and in particular, blood and blood serum. In some embodiments, the imaging agent has a solubility in 10 mM HEPES solution, pH 7.4, of at least about 10 μM. In further embodiments, the imaging agent has a solubility in 10 mM HEPES solution, pH 7.4, of at least about 15 μM at least about 20 μM, at least about 25 μM, at least about 30 μM, at least about 40 μM, or at least about 50 μM.

Certain embodiments of the imaging agent of the invention generally can be neutral molecules or salts. For example, if the dye or dye conjugate is charged, the imaging agent can be or contain a salt or acid (or combination thereof) of the dye or dye conjugate. For positively charged dyes or conjugates, suitable counter ions include anions such as fluoride, chloride, bromide, iodide, acetate, perchlorate, PF₆⁻, and the like. For negatively charged dyes or conjugates, suitable counterions include cations such as Na⁺, K⁺, and quaternary amines.

Certain embodiments of the imaging agent of the invention has significant improvements with regard to stability over time, allowing for dramatically improved operability and use for imaging and mapping. Similarly, the stability of the imaging agent allows for increased accuracy during surgery as the signal does not degrade over time.

The charge-balanced imaging agent of the is particularly advantageous because their behavior in vivo is believed to contribute to superior optical imaging properties. More specifically, the charge-balancing is believed to impart good biodistribution and clearance properties to the agents, and reduce undesirable non-specific binding. These in vivo properties help improve the target-to-background ratio of imaged cells and tissues, leading to higher resolution imaging. Furthermore, the charge-balanced imaging agents of the inventions are cleared primarily or exclusively by the kidneys.

In some embodiments, the tumor or cell is found in a subject. The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

### Dosage Forms and Administration Methods

In the methods of the invention, embodiments of the imaging agent can be administered at a predetermined dosage. The predetermined dosage amount is not particularly limited provided that the predetermined dosage is administered at least a minimum amount capable of being cleared by the kidneys within twelve hours.

In some embodiments, a detectably effective amount of the imaging agent of the presently disclosed methods is administered to a subject. In accordance with the presently disclosed subject matter, “a detectably effective amount” of the imaging agent is defined as an amount sufficient to yield an acceptable image using equipment which is available for clinical use. A detectably effective amount of the agent may be administered in more than one injection. The detectably effective amount of the imaging agent can vary according to factors such as the degree of susceptibility of the individual, the age, sex, and weight of the individual, idiosyncratic responses of the individual, the dosimetry, and instrument and film-related factors. Optimization of such factors is well within the level of skill in the art.

The administration of the agent of the invention can be by any means described herein and as generally acceptable to the patient and one of ordinary skill in the art. In particular, the administration of the charge-balanced imaging agent is intravenous.

The predetermined target amount is dependent on a variety of factors including, but not limited to the type of tumor or condition to be observed and the location of any cells desired to be imaged. As such, the predetermined amount is set by one of ordinary skill in the art prior to administration of the agent. Such factors include, but are not limited to, the determined dosage amount, the height, weight, age, body mass index, and gender of the patient or any combination thereof.

In particular embodiments, when the predetermined dosage amount is between about 2.5 mg and about 5.0 mg or greater, the predetermined target amount is 50% of the pre-determined dosage amount. In other embodiments, when the predetermined dosage amount is between about 0.5 mg and about 2.5 mg, the predetermined target amount is 60% of the pre-determined dosage amount. In still other embodiments, when the predetermined dosage amount is about 0.5 mg or less, the predetermined target amount is 80% of the pre-determined dosage amount.

The imaging agent of the invention can be administered by any suitable technique, including both enteral and parenteral methods. In some embodiments, the imaging agents can be formulated into pharmaceutically acceptable formulations and administered intravenously to an organism for imaging. The dosed organism can be imaged using, for example, a FLARE™ Image-Guided Surgery System, which is a continuous-wave (CW) intraoperative imaging system that is capable of simultaneous, real-time acquisition and display of color video (i.e., surgical anatomy) and two channels of invisible NIR fluorescent (700 nm and 800 nm) light. The imaging system can irradiate the dosed organism with radiation absorbed by the imaging agent, and detect optical signals, such as NIR fluorescence, emanating from the targeted portions of the organism containing the imaging agent. The detected signals can be recorded and analyzed by obtaining digital images or video of the subject organism, thereby facilitating diagnostic procedures and image-guided medical techniques.

Any route of administration may be suitable for administering the disclosed agents to a subject. In one embodiment, the disclosed imaging agents may be administered to the subject via intravenous injection. In another embodiment, the disclosed imaging agents may be administered to the subject via any other suitable systemic deliveries, such as parenteral, intranasal, sublingual, rectal, or transdermal administrations.

### APPLICATIONS, PROPERTIES, AND COMPOSITIONS

The conjugates described herein can be used for, e.g., planar optical, optical tomographic, endoscopic, photoacoustic, and sonofluorescent applications for the detection, imaging, and treatment of tumors and other abnormalities. The conjugates can also be used for localized therapy. This can be accomplished, e.g., by directing the conjugates to a desired target site, or allowing the conjugates to accumulate selectively in the target site; shining light of an appropriate wavelength to activate the agent. Thus, the new conjugates can be used to detect, image, and treat a section of tissue, e.g., a tumor.

In addition, the conjugates can be used to detect the presence of tumors and other abnormalities by monitoring the blood clearance profile of the conjugates, for laser assisted guided surgery for the detection of small micrometastases of, e.g., somatostatin subtype 2 (SST-2) positive tumors, and for diagnosis of atherosclerotic plaques and blood clots.

The conjugates can be formulated into diagnostic and therapeutic compositions for enteral or parenteral administration. Generally, these compositions contain an effective amount of the conjugate, along with conventional pharmaceutical carriers and excipients appropriate for the type of administration contemplated. For example, parenteral formulations include the conjugate in a sterile aqueous solution or suspension. Parenteral compositions can be injected directly into a subject at a desired site, or mixed with a large volume parenteral composition for systemic administration. Such solutions can also contain pharmaceutically acceptable buffers and, optionally, electrolytes, such as sodium chloride.

Formulations for enteral administration, in general, can contain liquids, which include an effective amount of the desired dye or dye conjugate in aqueous solution or suspension. Such enteral compositions can optionally include buffers, surfactants, and thixotropic agents. Compositions for oral administration can also contain flavoring agents, and other ingredients for enhancing their organoleptic qualities.

Generally, the diagnostic compositions are administered in doses effective to achieve the desired signal strength to enable detection. Such doses can vary, depending upon the organs or tissues to be imaged, and the imaging equipment being used. For example, Zeheer et al., Nature Biotechnology, 19, 1148-1154 (2001) uses 0.1 μmol/kg as a dose for IRDye78 conjugates in vivo. The diagnostic compositions can be administered to a patient systemically or locally to the organ or tissue to be imaged, and then the patient is subjected to the imaging procedure.

Generally, the conjugates or dye compounds absorb and emit light in the visible and infrared region of the electromagnetic spectrum, e.g., they can emit green, yellow, orange, red light, or near infrared light (“NIR”).

In some embodiments, the ZW-800-1, ZW-830-1, ZW-700-1-Forte dye emits and/or absorbs radiation having a wavelength from about 300 nm to about 1000 nm, e.g., from about 400 nm to about 900 nm, or from about 450 mu to about 850 nm. In particular embodiments, the ZW700-1 Forte dye emits and/or absorbs radiation having a wavelength of about 700 nm.

In some embodiments the conjugates and dye compounds have a maximum excitation and/or a maximum emission, measured in 10 mM HEPES solution, pH 7.4, of from about 525 mn to about 875 nm, e.g., from about 550 nm to about 825 nm, or from about 550 nm to about 800 nm.

The invention will be described in greater detail by way of specific examples. The following examples are offered for illustrative purposes and are not intended to limit the invention or claims in any manner. A variety of noncritical parameters in these examples can be changed or modified to yield essentially the same results.

### EXAMPLES

### Example 1: Imaging of Organisms

The FLARE™ Image-Guided Surgery System is a continuous-wave (CW) intraoperative imaging system that is capable of simultaneous, real-time acquisition and display of color video (i.e., surgical anatomy) and two channels of invisible NIR fluorescent (700 nm and 800 nm) light. Details of the theory, engineering, and operation of the imaging system has been described in detail previously. See, Tanaka, E., H. S. Choi, H. Fujii, M. G. Bawendi, and J. V. Frangioni, Image-guided oncologic surgery using invisible light completed: pre-clinical development for sentinel lymph node mapping. Ann Surg Oncol, 2006. 13: 1671-81; De Grand, A. M. and J. V. Frangioni, An operational near-infrared fluorescence imaging system prototype for large animal surgery. Technol Cancer Res Treat, 2003. 2: 553-562; and Nakayama, A., F. del Monte, R. J. Hajjar, and J. V. Frangioni, Functional near-infrared fluorescence imaging for cardiac surgery and targeted gene therapy. Molecular Imaging, 2002. 1: 365-377, each of which is incorporated herein by reference.

Specifications for the FLARE™ Image-Guided Surgery System is provided in Table 1 below.

TABLE 1
FLARE ™ NIR Fluorescence Imaging System Specifications

| Cate | Specification | Description |
|---|---|---|
| Physical | Size | Mobile Cart: 32″ W × 32″ D × 41.4″ H; Mast |
| | | Height: 82″ |
| | Weight | 675 lbs, including all electronics |
| | Arm | 6-degree-of-freedom; Reach: 43″-70″ from |
| | | floor, 50.7″ from cart |
| Electrical | Voltage and | 120 V AC, 60 Hz; single NEMA 5-15 120 V/15 |
| | Plug | A AC plug |
| | Current | 15 A max |
| | Grounding | Isolation transformer for all components; |
| | | redundant chassis grounding |
| | Leakage | <300 μA (per AAMI/IEC #60601) |
| Sterility | Shield | Disposable acrylic shield with ≥95% |
| | Drape | Disposable, custom-fit plastic drape bonded to |
| Light | Housing | Anodized aluminum with secondary 400 W |
| Source | | cooling plate |
| | Elements | Custom 25 mm circular LED arrays w/ |
| | Electronics | Custom passive and active boards with |
| | | embedded controller |
| | Fluence Rates | 40,000 lx white light (400-650 nm), 4 mW/cm² |
| | | of 700 nm (656-678 nm) excitation light, 14 mW/cm² of |
| Optics | Working | 18″ from surface of patient |
| | Field-of-View | 2.2 W × 1.7 H cm to 15 W × 11.3 cm (adjustable |
| | Emission/ | Color Video (400-650 nm), 700 nm fluorescence |
| | Reflectance | (689-725 nm), 800 nm fluorescence (800-848 nm), all |
| | Channels | |
| | Pixel | 640 × 480 for each camera |
| | System | 125 × 125 μm (x, y) to 625 × 625 μm (x, y) |
| | Display | Up to 15 Hz simultaneous acquisition on all 3 |
| | NIR Exposure | Adjustable from 100 μsec to 8 sec |
| Hands Free | Optics | Automatic zoom/focus |
| | Control | 6-pedal footswitch |
| Monitors | Number | 2 cart-mounted 20″ for operator; 1 satellite 20″ |
| | | on stand for surgeon |

### Example 3: In Vivo Characterization of Dyes and Conjugates

For in vivo characterization, 40 pmol/g (average 10 nmol) of a targeted dye of the invention can be injected IV into 25 g athymic nude mice harboring xenograft human tumors. The FLARE™ imaging system can be set to a 665 nm excitation fluence rate of 1 mW/cm2. Simultaneous color video and NIR fluorescence (700 nm) images can be acquired pre-injection, every 1 sec for the first 20 sec then every 1 min for 2 h. Camera acquisition can be held constant (typically 100 msec) and chosen to ensure that all intensity measurements are within the linear range of the 12-bit Orca-AG (Hamamatsu) NIR camera. Blood can be sampled at 0, 1, 2, 5, 10, 15, 30, 60, and 120 min via tail vein. Intensity-time curves for all major organs and tissues can be quantified. The peak fluorescence intensity and time can be determined for each tumor/tissue/organ, along with the intensity in each at 1 h post-injection.

### Example 2: In Vitro Optical and Stability Properties of Dyes

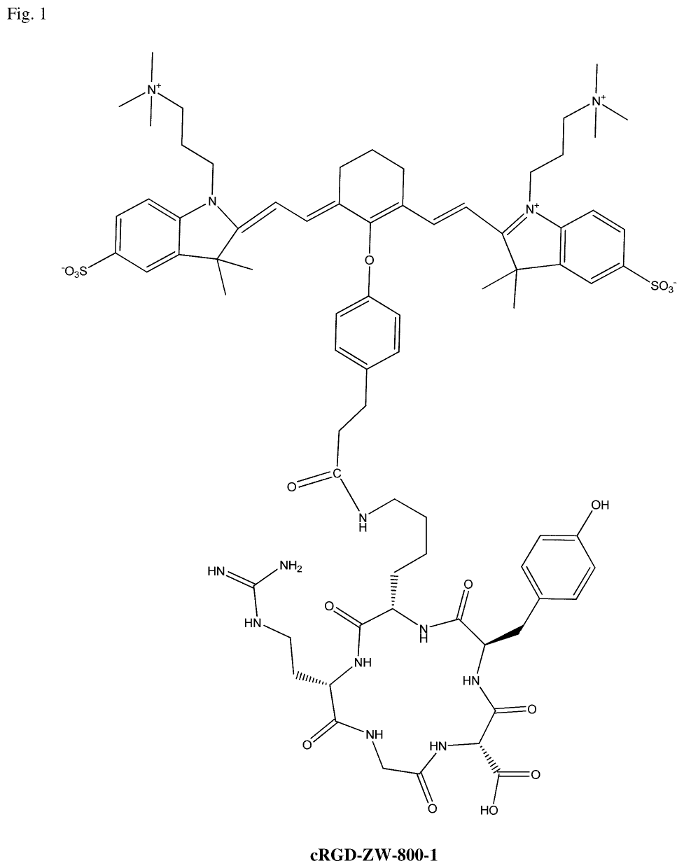

The targeted dyes of the invention are shown in FIG. 1 and are characterized with respect to their optical properties and stability in vitro. Commercial NIR fluorophores, such as IRDye™800-RS (RS-800), IRDye800-CW (CW-800), Cy5.5, and Cy7 have various degrees of sulfonation in order to achieve aqueous solubility.

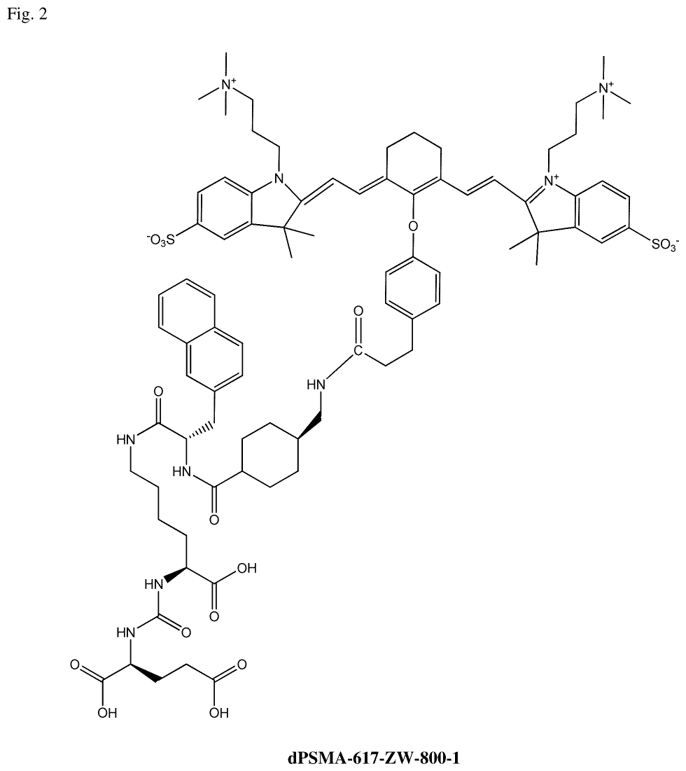

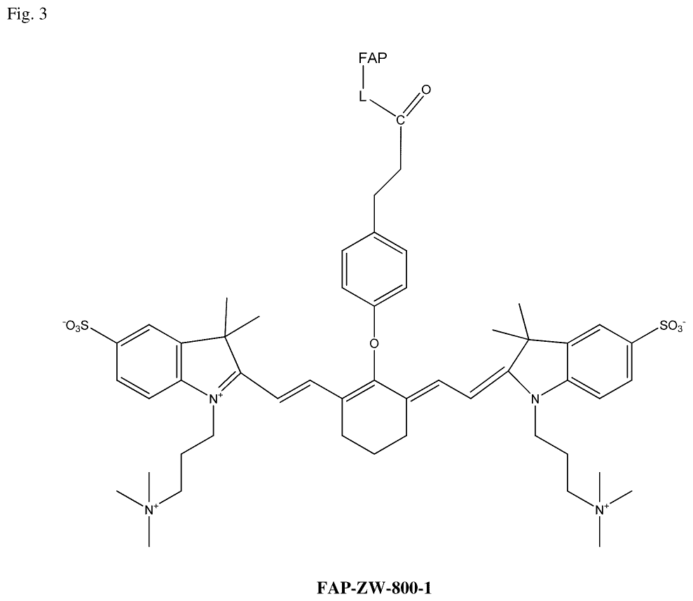

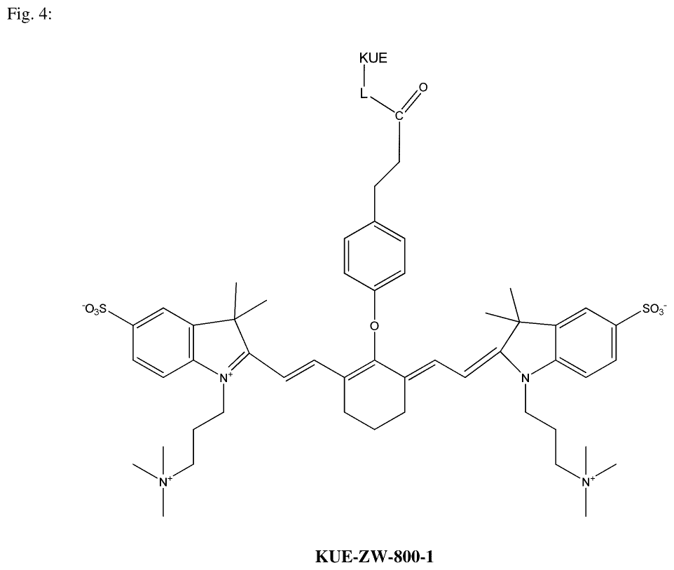

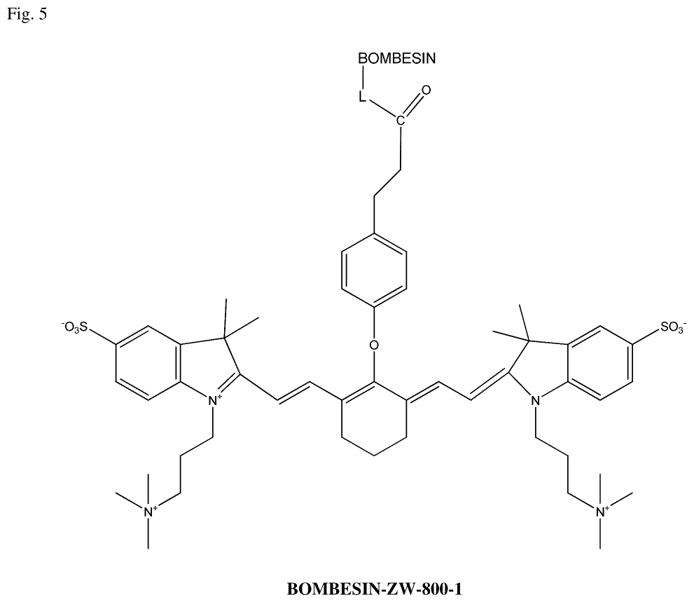

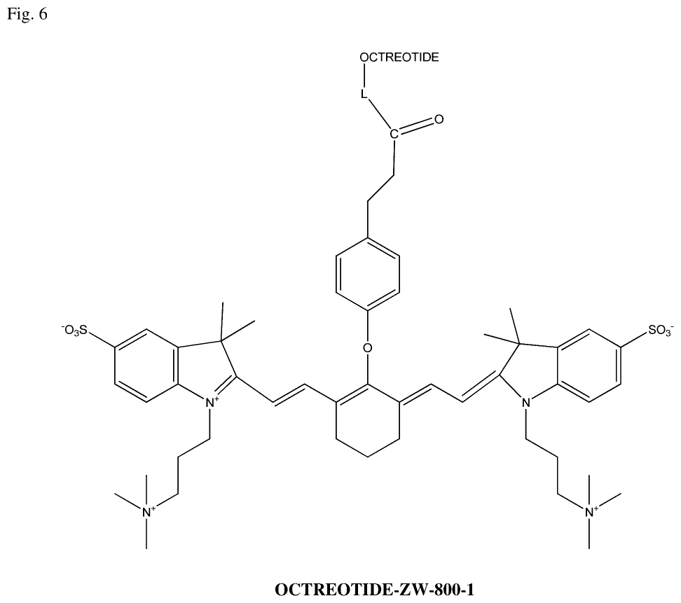

The normalized absorbance of ZW800-1 and ZW700-1c in 37° C. buffered FBS, pH 7.4 over time from 0 to 60 hours are shown in FIGS. 2-4. Note is made of the extreme stability of ZW700-1c compared to ZW800-1. Corresponding changes in absorption spectra during metabolism in warm serum is shown in FIGS. 5-6. Note is made of high stability of ZW700-1c over time, particularly as compared to ZW800-1.

Data generated in the same way is expected to show that dPSMA-617, KUE, FAP ligand, bombesin ligand, and octreotide, or their dimers, conjugated to ZW700-1 Forte will have further increased stability as compared to compared to cRGD-ZW800-1 or other ZW800-1 or ZW830-1 conjugates. ZW700-1 Forte conjugates should also retain their renal-predominant clearance, thus maximizing the rate of elimination of unbound dye from the body.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Those skilled in the art will recognize, or be able to ascertain using no more than routine experimentation, many equivalents to the specific embodiments of the invention described herein.

Such equivalents are intended to be encompassed by the following claims.

1. An imaging agent dye comprising a charge-balanced imaging agent conjugated to a targeting vector, the imaging agent dye having a labile linkage between the charge-balanced imaging agent and the targeting vector, and wherein the labile linkage stabilizes the imaging agent dye in an acidic environment of a target cell and allows for decomposition of the imaging agent dye in an environment external to the target cell to cause imaging agent dye internal to the target cell to fluoresce more than decomposed imaging agent dye external to the target cell thereby increasing a target cell to background ratio to 7.6 four hours after administration of the imaging agent dye.

2. The imaging agent dye according to claim 1 wherein the charge-balanced imaging agent is ZW-800-1, ZW-830-1, or ZW700-1 Forte.

3. The imaging agent dye according to claim 1 wherein the targeting vector is cRGD, dPSMA-617, KUE, a FAP binding vector, octreotide, or bombesin.

4. The imaging agent dye according to claim 1 wherein the charge-balanced imaging agent is conjugated to the targeting vector via a direct bond, or via a linking group.

5. The imaging agent dye according to claim 1 wherein the charge-balanced imaging agent conjugated to the targeting vector has the formula: 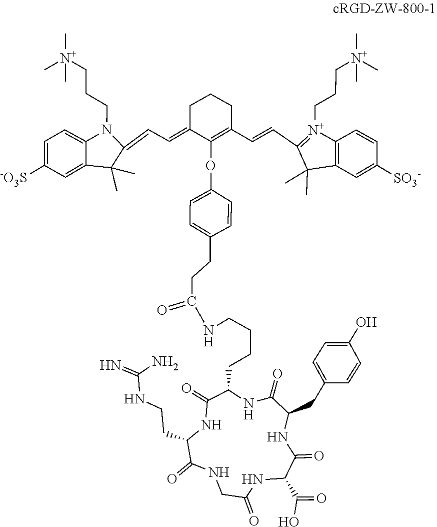
or a salt, solvate, or hydrate thereof;
or dimer thereof.

6. (canceled)

7. The imaging agent dye according to claim 1 wherein the charge-balanced imaging agent conjugated to the targeting vector has the formula: 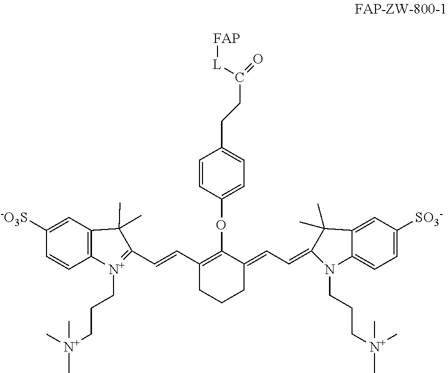
wherein L is an optional linking group;
or a salt, solvate, or hydrate thereof;
or a dimer thereof.

8. The imaging agent dye according to claim 1 wherein the charge-balanced imaging agent conjugated to the targeting vector has the formula: 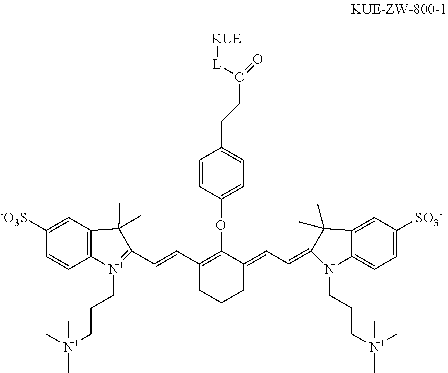
wherein L is an optional linking group;
or a salt, solvate, or hydrate thereof;
or dimer thereof.

9-10. (canceled)

11. The imaging agent dye according to claim 1 wherein the charge-balanced imaging agent conjugated to the targeting vector has the formula: 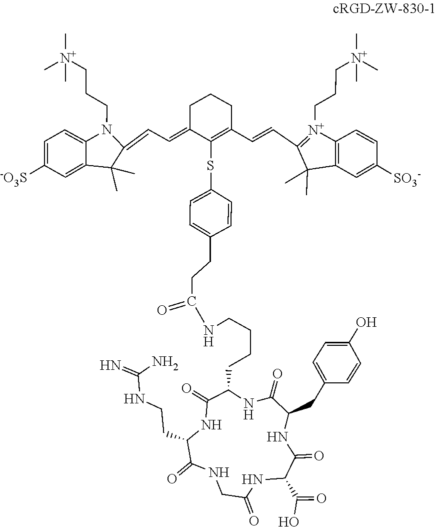
or a salt, solvate, or hydrate thereof;
or dimer thereof.

12. (canceled)

13. The imaging agent dye according to claim 1 wherein the charge-balanced imaging agent conjugated to the targeting vector has the formula: 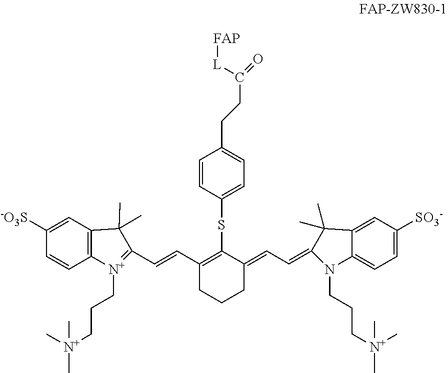
wherein L is an optional linking group;
or a salt, solvate, or hydrate thereof
or dimer thereof.

14. The imaging agent dye according to claim 1 wherein the charge-balanced imaging agent conjugated to the targeting vector has the formula: 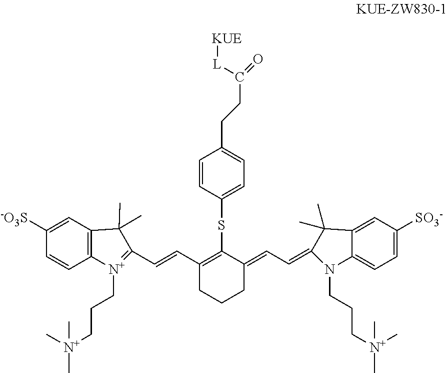
wherein L is an optional linking group;
or a salt, solvate, or hydrate thereof;
or dimer thereof.

15-16. (canceled)

17. The imaging agent dye according to claim 1 wherein the charge-balanced imaging agent conjugated to the targeting vector has the formula: 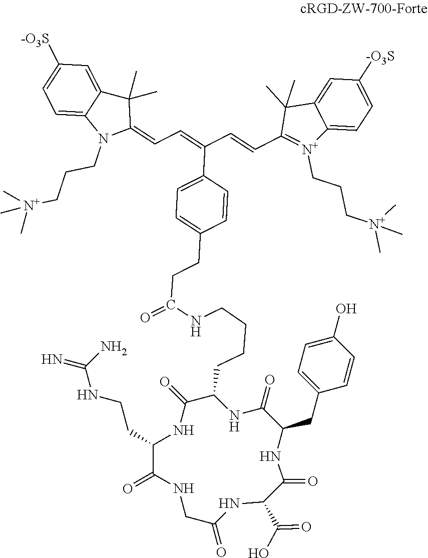
or a salt, solvate, or hydrate thereof;
or dimer thereof.

18. The imaging agent dye according to claim 1 wherein the charge-balanced imaging agent conjugated to the targeting vector has the formula: 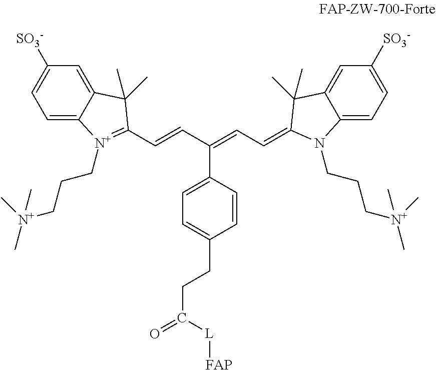
wherein L is an optional linking group;
or a salt, solvate, or hydrate thereof;
or dimer thereof.

19. (canceled)

20. The imaging agent dye according to claim 1 wherein the charge-balanced imaging agent conjugated to the targeting vector has the formula: 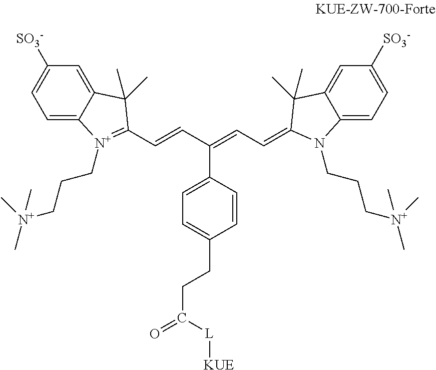
wherein L is an optional linking group;
or a salt, solvate, or hydrate thereof;
or dimer thereof.

21-22. (canceled)

23. A method of imaging tumor cells in a subject, the method comprising:
(a) administering an imaging effective amount of an imaging agent according to claim 1 to the subject,
(b) 2-4 hours after administration of the imaging agent, irradiating a region in the subject in which tumor cells are expected to be found with a wavelength absorbed by the imaging agent;
(c) and detecting a signal from the imaging agent, thereby imaging the tumor cells, wherein the target-to-background ratio four hours after administration is 7.6.

24-25. (canceled)

26. The method of claim 23, wherein the peak target-to-background ratio is observed 18-24 hours after administration.

27. The method of claim 23, wherein the subject is human.

28-31. (canceled)

32. The method of claim 23, wherein the imaging agent further comprises a radioisotope for either single-photon emission computed tomography (SPECT) or positron emission tomography (PET).

33. (canceled)

34. A method of treating cancer in a subject, the method comprising:
(a) administering an imaging effective amount of an imaging agent according to claim 1 to a subject,
(b) irradiating the cells, tissues or organs of a subject suspected of being cancerous at a wavelength absorbed by the imaging agent;
(c) diagnosing the cancer in the cells tissues, or organs of the subject by detecting a signal from the imaging agent; and
(d) administering a chemotherapeutic treatment, a radiotherapeutic treatment, or a surgical treatment to the subject to treat the cancer.

35. The imaging agent dye according to claim 1, wherein the target cell to background ratio is greater than 7.6 five to ten hours after administration of the imaging agent dye, and wherein the target cell to background ratio is 7.6 to 15 ten to twenty-four hours after administration, wherein the targeting vector is dPSMA-617, KUE, a FAP binding vector, octreotide, or bombesin.

36. The method of claim 23, wherein detecting the signal from the imaging agent, includes imaging the tumor cells, wherein the target-to-background ratio 5-10 hours after administration is greater than 7.6, and wherein the target cell to background ratio 10-24 hours after administration is 7.6 to 15, wherein the targeting vector is dPSMA-617, KUE, a FAP binding vector, octreotide, or bombesin.


SEQUENCE LISTING (6 sequences)

SEQ ID NO: 1 - protein, 9 aa, Homo sapiens
CGQKRTRGC

SEQ ID NO: 2 - protein, 12 aa, Homo sapiens
HTMYYHHYQHHL

SEQ ID NO: 3 - protein, 9 aa, Homo sapiens
CRKRLDRNC

SEQ ID NO: 4 - protein, 6 aa, Homo sapiens
CSPGAK

SEQ ID NO: 5 - protein, 8 aa, Homo sapiens
FCFWKTCT

SEQ ID NO: 6 - protein, 14 aa, Homo sapiens
XQRLGNQWAVGHLM